(19) United States
(12) Patent Application Publication
(10) Pub. No.: US 2005/0027323 A1
(43) Pub. Date: Feb. 3, 2005

(54) Implantable medical device for monitoring cardiac blood pressure and chamber dimension

(75) Inventors: Lawrence J. Mulligan, Andover, MN (US); Michael R.S. Hill, Minneapolis, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/000,973

(22) Filed: Oct. 30, 2001

(51) Int. Cl. A61N 1/365

(52) U.S. Cl. 607/18

(57) ABSTRACT

Implantable medical devices (IMDs) for monitoring signs of acute or chronic cardiac heart failure by measuring cardiac blood pressure and mechanical dimensions of the heart and providing multi-chamber pacing optimized as a function of measured blood pressure and dimensions are disclosed. The dimension sensor or sensors comprise at least a first sonomicrometer piezoelectric crystal mounted to a first lead body implanted into or in relation to one heart chamber that operates as an ultrasound transmitter when a drive signal is applied to it and at least one second sonomicrometer crystal mounted to a second lead body implanted into or in relation to a second heart chamber that operates as an ultrasound receiver. The ultrasound receiver converts impinging ultrasound energy transmitted from the ultrasound transmitter through blood and heart tissue into an electrical signal. The time delay between the generation of the transmitted ultrasound signal and the reception of the ultrasound wave varies as a function of distance between the ultrasound transmitter and receiver which in turn varies with contraction and expansion of a heart chamber between the first and second sonomicrometer crystals. One or more additional sonomicrometer piezoelectric crystal can be mounted to additional lead bodies such that the distances between the three or more sonomicrometer crystals can be determined. In each case, the sonomicrometer crystals are distributed about a heart chamber such that the distance between the separated ultrasound transmitter and receiver crystal pairs changes with contraction and relaxation of the heart chamber walls.

### CROSS-REFERENCE TO RELATED APPLICATION

Reference is hereby made to commonly assigned, co-pending U.S. patent application Ser. No. (P-7837.00) filed on even date herewith entitled IMPLANTABLE MEDICAL DEVICE EMPLOYING SONOMICROMETER OUTPUT SIGNALS FOR DETECTION AND MEASUREMENT OF CARDIAC MECHANICAL EVENTS in the names of Robert W. Stadler et al.

### FIELD OF THE INVENTION

The present invention relates generally to implantable medical devices (IMDs) for monitoring signs of acute or chronic cardiac heart failure and providing blood pressure and heart chamber dimension data to a physician to diagnose the condition of the heart and prescribe appropriate therapies including multi-chamber pacing optimized as a function of the measured blood pressure and heart chamber dimensions.

### BACKGROUND OF THE INVENTION

Patients suffering from chronic heart failure including congestive heart failure (CHF) manifest an elevation of left ventricular end-diastolic pressure, according to the well-known heterometric autoregulation principles espoused by Frank and Starling. This may occur while left ventricular end-diastolic volume remains normal due to a decrease in left ventricular compliance concomitant with increased ventricular wall stiffness. CHF due to chronic hypertension, ischemia, infarct or idiopathic cardiomyopathy is associated with compromised systolic and diastolic function involving decreased atrial and ventricular muscle compliance. These may be conditions associated with chronic disease processes or complications from cardiac surgery with or without specific disease processes. Most heart failure patients do not normally suffer from a defect in the conduction system leading to ventricular bradycardia, but rather suffer from symptoms which may include a general weakening of the contractile function of the cardiac muscle, attendant enlargement thereof, impaired myocardial relaxation and depressed ventricular filling characteristics in the diastolic phase following contraction. Pulmonary edema, shortness of breath, and disruption in systemic blood pressure are associated with acute exacerbations of heart failure.

All these disease processes lead to insufficient cardiac output to sustain mild or moderate levels of exercise and proper function of other body organs, and progressive worsening eventually results in cardiogenic shock, arrhythmias, electromechanical dissociation, and death. In order to monitor the progression of the disease and to assess efficacy of prescribed treatment, it is necessary to obtain accurate measures of the heart geometry, the degree of heart enlargement, and the mechanical pumping capability of the heart, e.g., ejection fraction, under a variety of metabolic conditions the patient is likely to encounter on a daily basis. These parameters are typically measured through the use of external echocardiogram equipment in the clinical setting. However, the measurement procedure is time consuming to perform for even a resting patient and cannot be practically performed replicating a range of metabolic conditions. Typically, the echocardiography procedure is performed infrequently and months or years may lapse between successive tests, resulting in a poor understanding of the progress of the disease or E no whether or not intervening drug therapies have been efficacious. Quite often, only anecdotal evidence from the patient is available to gauge the efficacy of the prescribed treatment.

Moreover, in many cases, diseased hearts exhibiting left ventricular dysfunction (LVD) and CHF also have conduction defects wherein cardiac depolarizations that naturally occur in one upper or lower heart chamber are not always conducted in a timely fashion either within the heart chamber or to the other upper or lower heart chamber. In such cases, the right and left heart chambers do not contract in optimum synchrony with each other, and cardiac output suffers due to the conduction defects. In addition, spontaneous depolarizations of the left atrium or left ventricle occur at ectopic foci in these left heart chambers, and the natural activation sequence is grossly disturbed. The natural electrical activation system through the heart involves sequential events starting with the sino-atrial (SA) node, and continuing through the atrial conduction pathways of Bachmann's bundle and internodal tracts at the atrial level, followed by the atrio-ventricular (AV) node, Common Bundle of His, right and left bundle branches, and final distribution to the distal myocardial terminals via the Purkinje fiber network. A common type of intra-atrial conduction defect is known as intra-atrial block (IAB), a condition where the atrial activation is delayed in getting from the right atrium to the left atrium. In left bundle branch block (LBBB) and right bundle branch block (RBBB), the activation signals are not conducted in a normal fashion along the right or left bundle branches respectively. Thus, in a patient with LBBB or RBBB, the activation of the ventricles is slowed, and the QRS is seen to widen due to the increased time for the activation to traverse the conduction path. For example, in a patient with LBBB, the delay in the excitation from the RV to the LV can be as high as 120 to 150 ms. Cardiac output deteriorates because the contractions of the right and left heart chambers are not synchronized sufficiently to eject the maximal blood volume. Furthermore, significant conduction disturbances between the right and left atria can result in left atrial flutter or fibrillation.

More particularly, as described in commonly assigned U.S. Pat. No. 6,129,744, patients suffering from LVD are also known to have elevated levels of catecholamines at rest because the body is attempting to increase cardiac output that induce a higher resting heart rate. In addition, the QT interval for such a patient is affected by the catecholamine level and thus has a changed pattern during exercise as well. These patients have a decreased QT response, or smaller change in QT, during exercise, such that the QT interval shortening during exercise is smaller than that found normally. Although QT interval is influenced independently by heart rate alone, as well as by exercise and catecholemines, it is not known to what extent each of these factors or both are responsible for the changed QT response to exercise in LVD patients. However, it is known that patients suffering LVD clearly have a different pattern of QT interval shortening during exercise. Moreover, the changed conductive patterns or a heart in heart failure are manifested by other changes in the PQRST waveforms, particularly an abnormally wide or long duration of the ventricular depolarization signal, or QRS.

These observed conduction defects have caused physicians to prescribe implantation of conventional, atrioventricular (AV) synchronous pacing systems, including DDD and DDDR pacing systems, marketed by Medtronic, Inc. and other companies, in certain patients for treatment of heart failure symptoms. Certain patient groups suffering heart failure symptoms with or without bradycardia tend to do much better hemodynamically with AV synchronous pacing due to the added contribution of atrial contraction to ventricular filling and subsequent contraction. However, fixed or physiologic sensor driven rate responsive pacing in such patients does not always lead to improvement in cardiac output and alleviation of the symptoms attendant to such disease processes because it is difficult to assess the degree of compromise of cardiac output caused by CHF and to determine the pacing parameters that are optimal for maximizing cardiac output, particularly the AV delay. Determining an optimal AV delay requires performing echocardiography studies or obtaining pressure data involving an extensive patient work-up as set forth in commonly assigned U.S. Pat. No. 5,626,623. Moreover, conventional DDD and DDDR pacemakers pace and sense only in the right atrium and right ventricle and cannot alleviate or alter IAB, LBBB, RBBB and QT interval widening.

Consequently, while some improvement has been reported in certain patients receiving two-chamber DDD or DDDR AV sequential pacemakers, the efficacy of the treatment is not established for larger patient populations. Therefore, efforts have been undertaken to develop more appropriate therapies, to identify patients who would benefit from such therapies, and to provide tools to assess the efficacy of the applied therapies.

A great deal of testing and data collection is necessary to obtain a thorough understanding of the heart failure condition and disease etiology of a symptomatic heart failure patient in order to prescribe any therapy, including drug therapies and IMD delivered stimulation therapies. Therefore, a number of other approaches have been proposed and advanced involving implantation of physiologic cardiac monitors for deriving and storing electrical EGM signals and mechanical performance indicating parameters over a prolonged time period and development of three and four-chamber pacing systems having the same capabilities.

An implantable EGM monitor for recording the cardiac electrogram from electrodes remote from the heart is disclosed in commonly assigned U.S. Pat. No. 5,331,966 and PCT publication WO 98/02209 and is embodied in the Medtronic® REVEAL® Insertable Loop Recorder having spaced housing EGM electrodes. More elaborate implantable hemodynamic monitors (IHMs) for recording the EGM from electrodes placed in or about the heart and other physiologic sensor derived signals, e.g., one or more of blood pressure, blood gases, temperature, electrical impedance of the heart and/or chest, and patient activity have also been proposed. The Medtronic® CHRONICLE® IHM is an example of such a monitor that is coupled through a lead of the type described in commonly assigned U.S. Pat. No. 5,564,434 having capacitive blood pressure and temperature sensors as well as EGM sense electrodes. Such implantable monitors when implanted in patients suffering from cardiac arrhythmias or heart failure accumulate date and time stamped data that can be of use in determining the condition of the heart over an extended period of time and while the patient is engaged in daily activities. A wide variety of other IMDs have been proposed to monitor many other physiologic conditions as set forth in U.S. Pat. No. 6,221,011

With respect to stimulation therapies other than DDD or DDDR pacing therapies, it was observed in the early days of implantable cardiac pacing that paired pacing (two or more closely spaced pacing pulses delivered at the time-out of an escape interval) and triggered or coupled pacing (one or more pacing pulses delivered following the detection of a P-wave or R-wave terminating an escape interval) with relatively short interpulse intervals (150 to 250 milliseconds in dogs and about 300 milliseconds in human subjects) beneficially slowed heart rate and increased cardiac output. The result of the second pulse, applied within the relative refractory period of the first paced or spontaneous depolarization, is to prolong the refractory period and effect a slowing of the heart rate from its spontaneous rhythm without an attendant mechanical myocardial contraction. This slowing effect has been employed since that time in many applications, including the treatment of atrial and ventricular tachycardias, where a single pulse or a burst of pulses are coupled to a spontaneous tachycardia event with a coupling interval that is shorter than and can be set as a fraction of the tachycardia interval as taught, for example, in U.S. Pat. Nos. 3,857,399 and 3,939,844. The slowing of the heart rate by coupled pacing is accompanied by the ability to increase or decrease the rate with subsequent coupled pacing within wide limits.

Paired and coupled stimulation of a heart chamber also cause a potentiation of contractile force effect through a phenomenon known as post-extrasystolic potentiation (PESP) described in detail in commonly assigned U.S. Pat. No. 5,213,098. The force of contraction of the heart is increased during the heart cycle that the paired or coupled stimulation is applied, and the increase persists but gradually diminishes over a number of succeeding heart cycles. Other measurable PESP effects that also persist but gradually decline over a number of heart cycles include changes in the peak systolic blood pressure, the rate of contraction of the ventricular muscle with a resulting increase of the rate of rise of intraventricular pressure (dP/dt), an increase in coronary blood flow, and an increase in the oxygen uptake of the heart per beat.

Various burst pulse stimulation regimens have been proposed for the treatment of heart failure including CHF that involve application of supra-threshold and/or sub-threshold stimulation paired or coupled pacing pulses or pulse trains. Moreover, various electrodes have been proposed for single site and multi-site delivery of the stimulation pulses to one or more heart chamber in the above-referenced patents and publications. However, it remains difficult to economically determine appropriate candidates that would benefit from such stimulation and to measure the efficacy of a given stimulation regimen and/or electrode array. Extensive catheterization procedures must be conducted of a heart failure patient to determine if he or she is a candidate for implantation of such a system. Then, the efficacy of any given treatment must be assessed at implantation and in periodic post-implant follow-up clinical tests. The patient work-up and follow-up testing must take into account or simulate known patient activities, patient posture, and whether the patient is awake or asleep in order to be representative of the heart failure condition over a daily time span

Consequently, determining the most efficacious burst stimulation parameters can be difficult and the results vary over time and due to a number of factors. Thus, widespread adoption of burst stimulation therapies for treating heart failure has not occurred.

A number of proposals have been advanced for providing pacing therapies to alleviate heart failure conditions and restore synchronous depolarization and contraction of a single heart chamber or right and left, upper and lower, heart chambers as described in detail in the above referenced '744 patent and in commonly assigned U.S. Pat. Nos. 5,403,356, 5,797,970 and 5,902,324, 6,219,579 and in U.S. Pat. Nos. 5,720,768 and 5,792,203. The proposals appearing in U.S. Pat. Nos. 3,937,226, 4,088,140, 4,548,203, 4,458,677, 4,332,259 are summarized in U.S. Pat. Nos. 4,928,688 and 5,674,259. The advantages of providing sensing at pace/sense electrodes located in both the right and left heart chambers is addressed in the '688 and '259 patents, as well as in U.S. Pat. Nos. 4,354,497, 5,174,289, 5,267,560, 5,514,161, and 5,584,867.

The medical literature also discloses a number of approaches of providing bi-atrial and/or bi-ventricular pacing as set forth in: Daubert et al., “Permanent Dual Atrium Pacing in Major Intra-atrial Conduction Blocks: A Four Years Experience”, PACE (Vol. 16, Part II, NASPE Abstract 141, p.885, April 1993); Daubert et al., “Permanent Left Ventricular Pacing With Transvenous Leads Inserted Into The Coronary Veins”, PACE (Vol. 21, Part II, pp. 239-245, January 1998); Cazeau et al., “Four Chamber Pacing in Dilated Cardiomyopathy”, PACE (Vol. 17, Part H, pp. 1974-1979, November 1994); and Daubert et al., “Renewal of Permanent Left Atrial Pacing via the Coronary Sinus”, PACE (Vol. 15, Part II, NASPE Abstract 255, p. 572, April 1992).

In most cases, it has been proposed that bi-ventricular pacing pulses be applied simultaneously to the right and left ventricles. An observation is made in commonly assigned U.S. Pat. No. 6,219,579 that the exact timing of mechanical events are important for properly controlling right and left heart chamber pacing so as to optimize left ventricular output. Specifically, it is known that actual contraction of one ventricular chamber before the other has the effect of moving the septum so as to impair full contraction in the later activated chamber. Thus, while concurrent or simultaneous pacing of the left and right ventricle may achieve a significant improvement for CHF patients, it is better to provide for pacing of the two ventricles in such a manner that the actual mechanical contraction of the left ventricle, with the consequent closing of the valve, occurs in a desired time relationship with respect to the mechanical contraction of the right ventricle and closing of the right value. For example, if conduction paths in the left ventricle are impaired, delivering a pacing stimulus to the left ventricle at precisely the same time as to the right ventricle may nonetheless result in left ventricular contraction being slightly delayed with respect to the right ventricular contraction.

In the above-referenced '324 patent, an AV synchronous pacing system is disclosed providing three or four heart chamber pacing through pace/sense electrodes located in or adjacent one or both of the right and left atrial heart chambers and in or adjacent to the right and left ventricular heart chambers. During an AV delay and during a V-A escape interval, a non-refractory ventricular sense event detected at either the right or left ventricular pace/sense electrodes starts a programmable conduction delay window (CDW) timer. A ventricular pace pulse is delivered to the other of the left or right ventricular pace/sense electrodes at the time-out of the CDW if a ventricular sense event is not detected at that site while the CDW times out. However, it is not always easy to determine just how to program the CDW duration to optimize the hemodynamics of the heart. As a consequence, it is important to provide a technique for measurement of mechanical events, such as a mechanical closure point of each of the ventricles, so as to be able to accurately program the sequence of pacing to achieve the desired dual ventricular pacing which optimizes ejection fraction, or cardiac output, for the individual patient.

Moreover, while such AV sequential, three or four-chamber pacing systems can be programmed to at least initially restore right and left and upper and lower heart synchrony in the clinical setting, they are not always able to maintain that synchrony over a range of heart rates and as the patient is exposed to other conditions of daily life including stress and exercise.

It is understood that the amount of blood being pumped by the heart is governed not only by the intrinsic or multi-chamber paced heart rate, but also by the stroke volume of the heart which is adversely lessened by heart failure. It has been recognized that it would be desirable to measure the contractility or displacement of the heart wall to determine the hemodynamic efficiency of the heart alone in an implanted monitor or in the context of controlling the operations of therapy delivery IMDs.

For example, the use an accelerometer positioned within a lead that is located within one of the chambers of the heart is disclosed in U.S. Pat. No. 5,549,650. The lead is attached to one of the walls of the heart so that movement of the wall of the heart causes the accelerometer that to develop an accelerometer signal that is processed to provide a first signal indicative of the contractility of the heart and a second signal indicative of the physical displacement of the wall of the heart. It is proposed in U.S. Pat. No. 4,730,619 to derive a measure of the ejection time of the ventricles, which is derived from the duration of contraction of the right ventricle which is determined from changes in right ventricular pressure. The right ventricular blood pressure is measured by a hermetically sealed absolute strain gauge transducer or a piezoresistive transducer mounted within a transvenous lead. The signals derived in the '650 and '619 patent are employed by the pacing system to adjust the pacing parameters to improve the hemodynamic efficiency of the heart as this information is directly related to the volume of blood being pumped by the heart during each ventricular contraction.

In an approach related to monitoring rejection of heart transplants, a magnetic field responsive Hall effect device and a permanent magnet are implanted directly across the septum or a heart wall as taught in U.S. Pat. No. 5,161,540, and the Hall effect device is powered by an implantable generator and telemetry transceiver. The compliance of the heart wall is monitored to detect any loss of compliance characteristic of rejection of the heart transplant is transmitted from the implanted system.

A discussion of a wide number of mechanical and electrical parameter sensors employed in the art to assess cardiac functions and hemodynamic efficiency is set forth in U.S. Pat. No. 5,243,976. In the '976 patent, continuous wave (CW) and pulsed wave (PW) Doppler emitters are incorporated into pacing leads to measure blood flow, and the flow measurements are employed to regulate atrial and ventricular pacing parameters and for other purposes.

In the above-referenced '579 patent, impedance measurements are made in or across the heart chambers from which accurate timing signals are obtained reflecting mechanical actions, e.g., valve closures, so that accurate timing information is available for controlling electrical activation and resultant mechanical responses for the respective different heart chambers. The impedance or mechanical sensing determinations are preferably made by multiplexing through fast switching networks to obtain the desired impedance measurements in different heart chambers. In a preferred embodiment, control of left heart pacing, is based primarily upon initial detection of a spontaneous signal in the right atrium, and upon sensing of mechanical contraction of the right and left ventricles. In a heart with normal right heart function, the right mechanical AV delay is monitored to provide the timing between the initial sensing of right atrial activation (P-wave) and right ventricular mechanical contraction. The left heart is controlled to provide pacing which results in left ventricular mechanical contraction in a desired time relation to the right mechanical contraction; e.g., either simultaneous or just preceding the right mechanical contraction; cardiac output is monitored through impedance measurements, and left ventricular pacing is timed to maximize cardiac output. In patients with IAB, the left atrium is paced in advance of spontaneous depolarization, and the left AV delay is adjusted so that the mechanical contractions of the left ventricle are timed for optimized cardiac output from the left ventricle.

The '579 patent also sets forth algorithms using the impedance measurements to obtaining and storing data reflecting heart failure state and for optimizing bi-ventricular pacing to provide maximum cardiac output.

A CHF monitor/stimulator is disclosed in commonly assigned U.S. Pat. No. 6,104,949 that senses the trans-thoracic impedance as well as patient posture and provides a record of same to diagnose and assess the degree and progression of CHF. The sensed trans-thoracic impedance is dependent on the blood or fluid content of the lungs and assists in the detection and quantification of pulmonary edema symptomatic of CHF. Trans-thoracic impedance is affected by posture, i.e. whether the subject is lying down or standing up, and the sensed trans-thoracic impedance is correlated to the output of the patient posture detector to make a determination of presence of and the degree of pulmonary edema for therapy delivery and/or physiologic data storage decisions.

A monitor/stimulator is disclosed in U.S. Pat. No. 5,417,717 that monitors and assesses level of cardiac function then permits a physician to arbitrate the therapy mode, if therapy is indicated. The monitor stimulator assesses impedance, EGM, and/or pressure measurements, and then calculates various cardiac parameters. The results of these calculations determine the mode of therapy to be chosen. Therapy may be administered by the device itself or a control signal may be telemetered to various peripheral devices aimed at enhancing the heart's function. Alternatively, the device may be programmed to monitor and either store or telemeter information without delivering therapy. One suggested therapy comprises delivery or AV synchronous, bi-ventricular pacing pulses to the heart.

Particularly, the implantable monitor/stimulator of the '717 patent monitors conventional parameters of cardiac function and contractile state, including all phases of the cardiac cycle. Thus, assessments of contractile state measured include indices of both cardiac relaxation and contraction. Utilizing the dual source ventricular impedance plethysmography technique described in U.S. Pat. No. 4,674,518, the monitor/stimulator monitors cardiac function by assessing hemodynamic changes in ventricular filling and ejection or by calculating isovolumic phase indices by known algorithms. The primary calculations involve: (1) the time rate of change in pressure (dP/dt) or volume (dV/dt) as isovolumic indicators of contractility; (2) ejection fraction as an ejection phase index of cardiac function according to the known quotient of stroke volume divided by end diastolic volume; (3) Maximal elastance, EM; (4) regression slope through maximal pressure-volume points as a further ejection phase index of contractility using the method of Sagawa; (5) stroke work according to the known pressure-volume integration; (6) the time course of minimum (end) diastolic pressure-volume measurements according to the method of Glantz as a measure of diastolic function; and (7) cardiac output calculation according to the known product of heart rate and stroke volume as an index of level of global function.

While measurement and storage of this group of parameters of cardiac function and contractile state can provide valuable information about the state of heart failure, the sensors are not always easy to implant so that they perform reliably chronically and under the range of conditions encountered by the patient and resulting from progression of the heart failure. The proposed systems employing locally * disposed accelerometers at one or more location in the heart or distributed impedance measuring electrodes to detect and measure heart motion and to derive the above-described parameters are difficult to implement and subject to outside influences that distort the signals.

Chronically collected data from patients with heart failure is needed so that the treating cardiologist can properly and accurately chart the progression, determine the nature of the heart failure, and be able to implement the optimal treatment in a timely fashion. There is a substantial need in the art for a pacemaker or other IMD having the capacity to identify the progression or remission of heart failure and to provide such indication to the patient's physician so that options can be assessed from time to time to treat the changing patient condition.

Given the demonstrated feasibility of PESP and four-chamber cardiac pacing, and the availability of techniques for sensing natural cardiac signals and mechanical events, there nonetheless remains a need for developing a system which is adapted to obtain valuable data and to make changes in the pacing parameters to optimize mechanical performance of the heart. There is a need for such an IMD providing bi-ventricular and/or bi-atrial pacing wherein the pacing rate and A-A delay or V-V delay as well as the AV delay are periodically optimized by the IMD operating system to provide appropriate hemodynamic status during various ambulatory conditions and activities of daily living using cardiac pressures, dimensions and wall displacement.

### SUMMARY OF THE INVENTION

In view of the above need, the present invention provides a system and method for monitoring patient cardiac signals and processing such signals within an IMD to provide data from which the onset or progression of heart failure can be determined. It is to be understood that the invention is applicable to various forms of heart failure, including left heart conduction disorders such as IAB, LBBB and RBBB, and other forms of heart dysfunction including LVD.

In accordance with the present invention, an implantable stimulator and monitor measures a group of parameters indicative of the state of heart failure employing EGM signals, measures of blood pressure including absolute pressure P, i developed pressure DP (DP=systolic P−diastolic P), and/or dP/dt, and measures of heart chamber dimension (D) over one or more cardiac cycles to derive trend data indicative of the state of heart failure. The measures of pressure and dimension developed over heart cycles can also be employed in pressure-dimension relationship analysis to provide other useful information about the status of the cardiac function.

The dimension sensor or sensors comprise at least a first sonomicrometer piezoelectric crystal mounted to a first lead body implanted into or in relation to one heart chamber, e.g., the RV, that operates as an ultrasound transmitter when a drive signal is applied to it and at least one second sonomicrometer crystal mounted to a second lead body implanted into or in relation to a second heart chamber, e.g., the LV, the LA or the RA, that operates as an ultrasound receiver. The ultrasound receiver converts impinging ultrasound energy transmitted from the ultrasound transmitter through blood and heart tissue into an electrical signal. The time delay between the generation of the transmitted ultrasound signal and the reception of the ultrasound wave varies as a function of the distance between the ultrasound transmitter and receiver which in turn varies with contraction and expansion of a heart chamber between the first and second sonomicrometer crystals. One or more additional sonomicrometer piezoelectric crystal can be mounted to additional lead bodies such that the distances between the three or more sonomicrometer crystals can be determined. In each case, the sonomicrometer crystals are distributed about a heart chamber such that the distance between the separated ultrasound transmitter and receiver crystal pairs changes with contraction and relaxation of the heart chamber walls whereby the instantaneous measured distance is characterized as, or is proportional to, the instantaneous heart chamber dimension D.

The instantaneous heart chamber dimension (D) is an indicator of the instantaneous heart chamber volume (V) and can be employed in pressure dimension relationship analyses akin to pressure-volume relationship analyses. More than one receiver crystal can be positioned about a given heart chamber, e.g., the LV, and paired with a transmitter crystal to derive sets of dimension data from which heart chamber volume V may be more closely extrapolated.

A heart failure parameter of interest comprises end systolic elastance (E_{ES}), i.e., the ratio of end systolic blood pressure P to an end systolic volume V or dimension D of a heart chamber and the end-diastolic elastance (E_{ED}). The E_{ES} and E_{ED} heart failure state parameter is determined and stored periodically when patient posture, activity level, intrinsic heart rate, and regularity are within programmable ranges. The E_{ES} and E_{ED} parameter data is associated with a date and time stamp and with other patient data, e.g., patient activity level, and the associated parameter data is stored in IMD memory for retrieval at a later date employing conventional telemetry systems. Incremental changes in the parameter data over time, taking any associated time of day and patient data into account, provide a measure of the degree of change in the CHF condition of the heart.

The sonomicrometer distance and pressure sensing system and method of the present invention has particular application to the derivation of LV pressure and dimension data and the development of the E_{ES} and E_{ED} data that provide a global metric of heart failure status and remodeling that occurs due to the pathophysiology. In general terms, as the heart chamber dimension D and volume V increase and pressure P decreases or remains the same, the E_{ES} decreases and the E_{ED} increases. This is the common observation as the heart failure worsens. The data also provides a global metric of heart failure status and severe remodeling that occurs during delivery of drug and/or stimulation therapies. In general terms, an effective therapy leading to an improvement in the heart failure state is indicated by a reduction in the heart chamber dimension D and volume V, pressure P increases or remains the same and E_{ES} increases while E_{ED} decreases.

The percent systolic shortening provides additional information which can be used to evaluate the AV and VV pacing intervals. Percent systolic shortening is measured by dividing the difference of the dimensions at end-systole and end-diastole by the end-diastolic value. The amount of shortening occurring each beat is stable and decreases as the amount of ventricular dysfunction increases.

The implantable stimulator and monitor that is capable of performing these functions comprises an implantable pulse generator (IPG) or monitor and lead system extending into operative relation with at least one and preferably multiple heart chambers for electrical sensing and stimulation, blood pressure measurement and chamber volumetric measurement during contraction and relaxation. The IPG/monitor has a sense amplifier for each heart chamber of interest that is coupled through a lead conductor with electrical stimulation/sense electrodes for sensing cardiac electrical heart signals originating in or traversing that heart chamber so that the sense amplifier can detect a P-wave in an atrial chamber or R-wave in a ventricular chamber.

Preferably an IPG is provided having timing circuitry for timing out atrial and/or ventricular escape intervals and the ESI of coupled or paired PESP stimulating pulse(s) and a pulse generator coupled with at least one stimulation/sense electrode for delivering pacing pulses and PESP stimulation pulses to each heart chamber of interest. The IPG has blood pressure signal processing circuitry coupled through lead conductors with a blood pressure sensor located in a distal lead section in or in operative relation to each heart chamber of interest for deriving blood pressure P and dP/dt samples. The IPG also has dimension D and volume V determining circuitry coupled with one or more of the sonomicrometer dimension sensors located in or in relation with each heart chamber of interest for deriving a signal representative of heart chamber dimension D and volume V.

In order to overcome the disadvantages and limitations of previously known approaches for optimizing pacing therapy, the processing system of the present invention processes the derived pressure and dimension to produce signals representative of stroke volume, percent systolic shortening, stroke work, cardiac contractility, pre-ejection period, filling time and ejection time. These signals are used to provide hemodynamically optimal pacing therapy while the patient is at rest and to provide hemodynamically optimal rate-responsive pacing therapy. Stroke volume, percent systolic shortening, stroke work, cardiac contractility, pre-ejection period, filling time and ejection time may be used, individually or together in combination, to adjust the parameters of the implantable cardiac stimulating device so that hemodynamically optimal pacing therapy may be provided.

The pressure and dimension signals as provided by the processing system of the present invention have been found to be related to stroke work. To illustrate, pressure and dimension signals from a patient suffering from dilated cardiomyopathy demonstrate a reduced pulse pressure change and a reduced dimensional change (volume change) during a cardiac cycle. Note that both absolute pressure and overall dimension may be increased over long time periods, yet the change is attenuated. This indicates that the total volume of blood being pumped by the heart during each heartbeat is abnormal.

The present invention is directed to a processing system which processes the pressure and dimension signals to determine cardiac stroke volume, percent systolic shortening, stroke work, cardiac contractility, pre-ejection period, filling time and ejection time, and then use these calculated values to optimize the timing of the stimulation provided to the patient by the rate-responsive pacemaker. In this manner, operational parameters of the rate-responsive pacemaker may be adjusted, in a closed loop manner, as the circumstances for optimal hemodynamic performance change. For example, the rate-responsive pacemaker may continually adjust the heart rate of the patient to provide hemodynamically optimal pacing therapy, thereby substantially maximizing cardiac output during periods of metabolic need.

The present invention initially establishes optimal values for heart rate, A-A, V-V and AV delays. Then, for each optimization cycle, cardiac performance is measured using pressure and dimension signals for selected combinations of heart rate, A-A, V-V and AV delays. The interval values resulting in the greatest measured cardiac performance become the new optimal values for the next cycle.

In another aspect of the present invention, methods for providing hemodynamically optimal rate-responsive pacing therapy and hemodynamically optimal pacing therapy at rest are described. The methods of providing hemodynamically optimal pacing therapy (for rate-response or at rest) may utilize, individually or in combination, stroke volume, percent systolic shortening, stroke work, cardiac contractility, pre-ejection period, filling time and ejection time to optimize cardiac performance.

This summary of the invention and the objects, advantages and features thereof have been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic diagram depicting a multi-channel, atrial and bi-ventricular, monitoring/pacing IMD in which the present invention is preferably implemented employing distributed sonomicrometer piezoelectric crystals to derive dimension signals during systolic and diastolic heart contraction phases;

FIG. 2 is a simplified block diagram of one embodiment of IMD circuitry and associated leads employed in the system of FIG. 1 enabling selective therapy delivery and/or monitoring in one or more heart chamber;

FIG. 3 is a simplified block diagram of a multi-chamber measurement system for deriving RV pressure signals, dimension measurements and cardiac EGM signals employed in monitoring CHF and optionally pacing the heart and delivering pacing therapy in accordance with the present invention;

FIG. 4 is a comprehensive flow-chart illustrating the operating modes of the IMD circuitry of FIG. 3 in a variety of AV synchronous, bi-ventricular pacing modes in accordance with one embodiment of the invention;

FIG. 5 is a flow chart illustrating the steps of delivering ventricular pace pulses following time-out of an AV delay in FIG. 4;

FIG. 6A-6B is a flow chart illustrating the steps of delivering ventricular pace pulses following a ventricular sense event during the time-out of an AV delay or the V-A escape interval in FIG. 4;

FIG. 7 is a flow chart illustrating the steps of periodically operating the system of FIG. 3 to derive RV pressure signals, dimension measurements and cardiac EGM signals, storing the signals, optionally processing the signals to update pacing timing parameters, and telemetering the stored data and updated parameters to an external programmer;

FIG. 8 is a flow chart illustrating the steps of operating the system of FIG. 3 to derive RV pressure signals and dimension measurements and processing the signals to provide elastance data in step S416 of FIG. 7;

FIG. 9 is a graphical depiction of measured left ventricular PV loops during a modification of preload with end systolic PV points shown at the upper left;

FIG. 10 is a graphical depiction of a linear regression of the end systolic PV points of FIG. 18 to derive the slope of the LV E_{ES};

FIG. 11 is a graphical depiction of measured left ventricular PV loops during normal heart function with end systolic PV points shown at the upper left;

FIG. 12 is a graphical depiction of a linear regression of the end systolic PV points of FIG. 20 wherein the determination of slope of the LV E_{ES} is not reliable;

FIG. 13 is a flow chart illustrating the steps of employing elastance parameter data derived in FIGS. 7 and 8 at differing temporary settings of pacing parameters to derive the set of pacing parameters providing optimal right and left mechanical heart function;

FIG. 14 depicts the relationship of heart chamber EGM, pressure, flow, and volume during a heart cycle; and

FIG. 15 is a flow chart illustrating an alternative manner of deriving pacing parameter values from diagnostic values derived from measured pressure and distance signals that optimize right and left heart mechanical heart function

### DETAILED DESCRIPTION OF THE PREFERRED EMBODIMENTS

In the following detailed description, references are made to illustrative embodiments for carrying out the invention. It is understood that other embodiments may be utilized without departing from the scope of the invention. For example, the invention is disclosed in detail herein in the context of an AV sequential, three chamber or four chamber, pacing system operating in demand, atrial tracking, and triggered pacing modes for restoring synchrony in depolarizations and contraction of left and right ventricles in synchronization with atrial sensed and paced events for treating heart failure and/or bradycardia in those chambers. This embodiment of the invention is programmable to operate as a three or four chamber pacing system having an AV synchronous operating mode for restoring upper and lower heart chamber synchronization and right and left atrial and/or ventricular chamber depolarization synchrony.

It should be appreciated that the present invention may be utilized in an implantable monitor to gather data in patients suffering various forms of heart failure. The system of the present invention may also may be incorporated into an anti-tachyarrhythmia system including specific high rate pacing and cardioversion shock therapies for providing staged therapies to treat a diagnosed tachyarrhythmia.

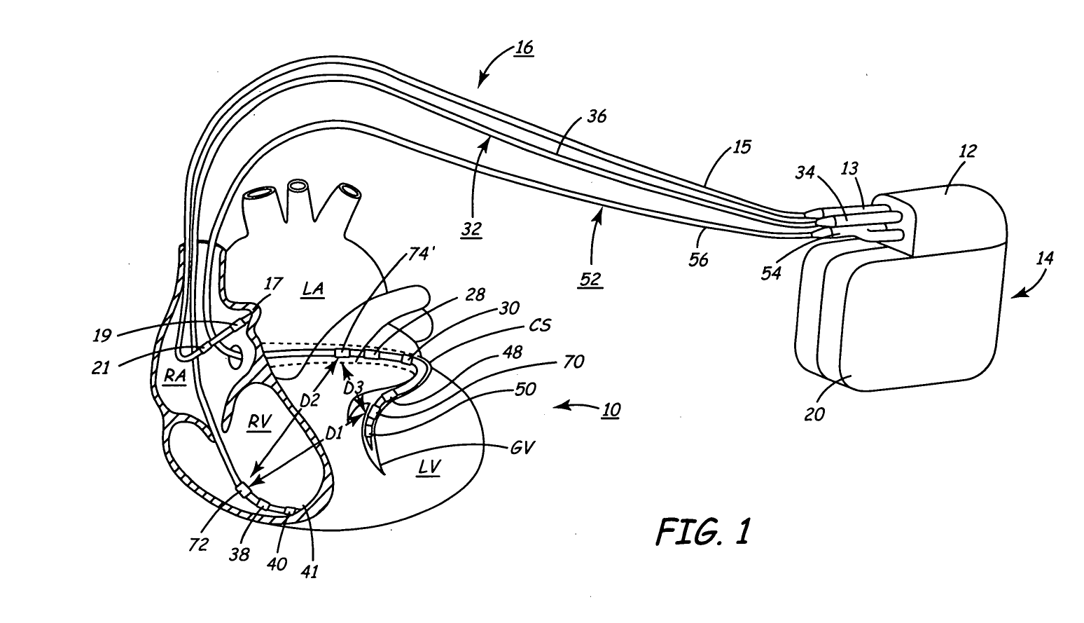

In FIG. 1, heart 10 includes the upper heart chambers, the right atrium (RA) and left atrium (LA), and the lower heart chambers, the right ventricle (RV) and left ventricle (LV) and the coronary sinus (CS) extending from the opening in the right atrium laterally around the atria to form the great vein (GV) that extends further inferiorly into branches of the GV. FIG. 1 is an illustration of transmission of the cardiac depolarization waves through the RA, LA, RV and LV in a normal electrical activation sequence at a normal heart rate with the conduction times exhibited thereon in seconds. The cardiac cycle commences normally with the generation of the depolarization impulse at the SA Node in the right atrial wall and its transmission through the atrial conduction pathways of Bachmann's Bundle and the Internodal Tracts at the atrial level into the left atrial septum. The RA depolarization wave reaches the atrio-ventricular (AV) node and the atrial septum within about 40 msec and reaches the furthest walls of the RA and LA within about 70 msec, and the atria complete their contraction as a result. The aggregate RA and LA depolarization wave appears as the P-wave of the PQRST complex when sensed across external ECG electrodes and displayed. The component of the atrial depolarization wave passing between a pair of unipolar or bipolar pace/sense electrodes, respectively, located on or adjacent the RA or LA is also referred to as a sensed P-wave. Although the location and spacing of the external ECG electrodes or implanted unipolar atrial pace/sense electrodes has some influence, the normal P-wave width does not exceed 80 msec in width as measured by a high impedance sense amplifier coupled with such electrodes. A normal near field P-wave sensed between closely spaced bipolar pace/sense electrodes and located in or adjacent the RA or the LA has a width of no more than 60 msec as measured by a high impedance sense amplifier.

The depolarization impulse that reaches the AV Node is distributed inferiorly own the bundle of His in the intraventricular septum after a delay of about 120 msec. The depolarization wave reaches the apical region of the heart about 20 msec later and is then travels superiorly though the Purkinje Fiber network over the remaining 40 msec. The aggregate RV and LV depolarization wave and the subsequent T-wave accompanying re-polarization of the depolarized myocardium are referred to as the QRST portion of the PQRST cardiac cycle complex when sensed across external ECG electrodes and displayed. When the amplitude of the QRS ventricular depolarization wave passing between a bipolar or unipolar pace/sense electrode pair located on or adjacent the RV or LV exceeds a threshold amplitude, it is detected as a sensed R-wave. Although the location and spacing of the external ECG electrodes or implanted unipolar ventricular pace/sense electrodes has some influence, the normal R-wave width does not exceed 80 msec in width as measured by a high impedance sense amplifier. A normal near field R-wave sensed between closely spaced bipolar pace/sense electrodes and located in or adjacent the RV or the LV has a width of no more than 60 msec as measured by a high impedance sense amplifier.

The typical normal conduction ranges of sequential activation are also described in the article by Durrer et al., entitled “Total Excitation of the Isolated Human Heart”, in CIRCULATION (Vol. XLI, pp. 899-912, June 1970). This normal electrical activation sequence becomes highly disrupted in patients suffering from advanced CHF and exhibiting IACD, LBBB, RBBB, and/or IVCD. These conduction defects exhibit great asynchrony between the RV and the LV due to conduction disorders along the Bundle of His, the Right and Left Bundle Branches or at the more distal Purkinje Terminals. Typical intra-ventricular peak—peak asynchrony can range from 80 to 200 msec or longer. In RBBB and LBBB patients, the QRS complex is widened far beyond the normal range to from >120 msec to 250 msec as measured on surface ECG. This increased width demonstrates the lack of synchrony of the right and left ventricular depolarizations and contractions.

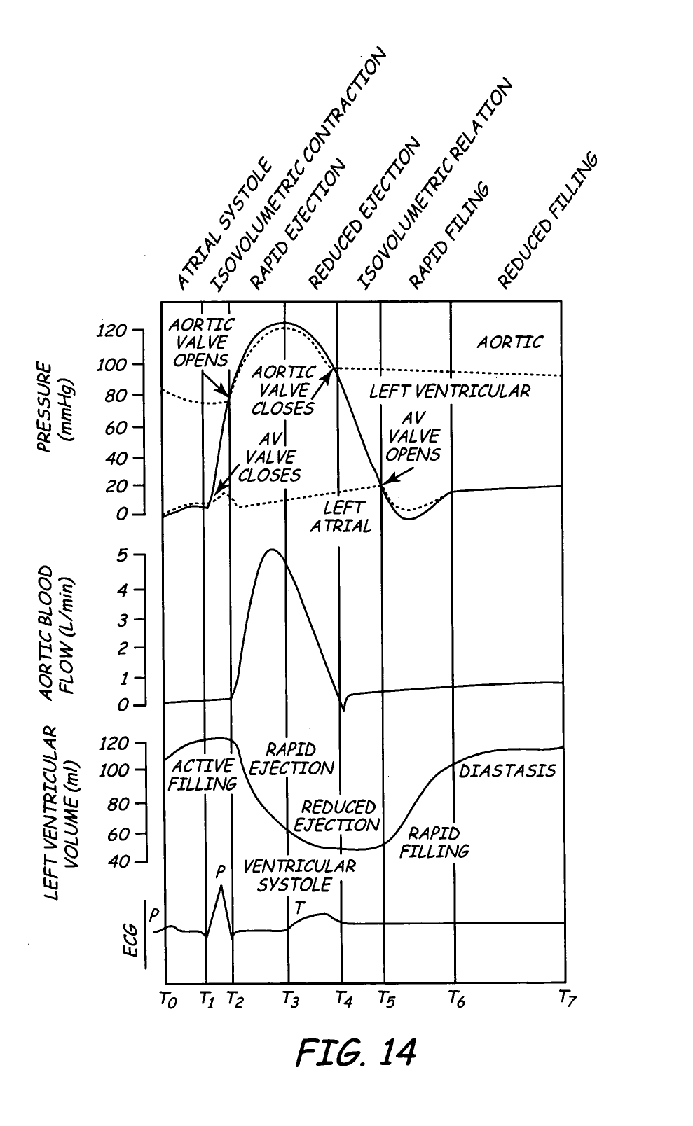

FIG. 14 depicts the relationship of heart chamber EGM, pressure, flow, and volume during a heart cycle reproduced from the above-referenced '464 patent which depicts the electrical depolarization waves attendant a normal sinus rhythm cardiac cycle in relation to the fluctuations in absolute blood pressure, aortic blood flow and ventricular volume in the left heart. The right atria and ventricles exhibit roughly similar pressure, flow and volume fluctuations, in relation to the PQRST complex, as the left atria and ventricles. It is understood that the monitoring and stimulation therapy aspects of this invention may reside and act on either or both sides of the heart. The cardiac cycle is completed in the interval between successive PQRST complexes and following relaxation of the atria and ventricles as the right and left atria re-fill with venous blood and oxygenated blood. In sinus rhythm, the interval between depolarizations may be on the order of 500.0 ms to 1,000.0 ms for a corresponding sinus heart rate of 120 bpm to 60 bpm, respectively. In this time interval, the atria and ventricles are relaxed, and overall atrial size or volume may vary as a function of pleural pressure and respiration. In the blood pressure diagrams of FIG. 14, it may be observed that the atrial and ventricular blood pressure changes track and lag the P-waves and R-waves of the cardiac cycle. The time period T₀-T₁ encompasses the AV delay.,

In patients suffering from cardiac insufficiency arising from bradycardia due to an incompetent SA node or AV-block, atrial and/or ventricular conventional pacing may be prescribed to restore a sufficient heart rate and AV synchrony. In FIG. 14, for example, atrial and/or ventricular pacing pulses would precede the P-wave and the deflection of the QRS complex commonly referred to as the R-wave. Cardiac output may be reduced by the inability of the atrial or ventricular myocardial cells to relax following atrial (T₀-T₁) and ventricular (T₂-T₄) systolic periods. Prolonged systolic time periods reduce passive filling time T₄ -T₇ as shown in FIG. 14. Thus, the amount of blood expelled from the atria and/or ventricles in the next cardiac cycle may be less than optimum. This is particularly the case with CHF patients or other patients in whom the stiffness of the heart is increased, cardiac filling during the passive filling phase (T₄ -T₇) and during atrial systole (T₀-T₁) is significantly limited.

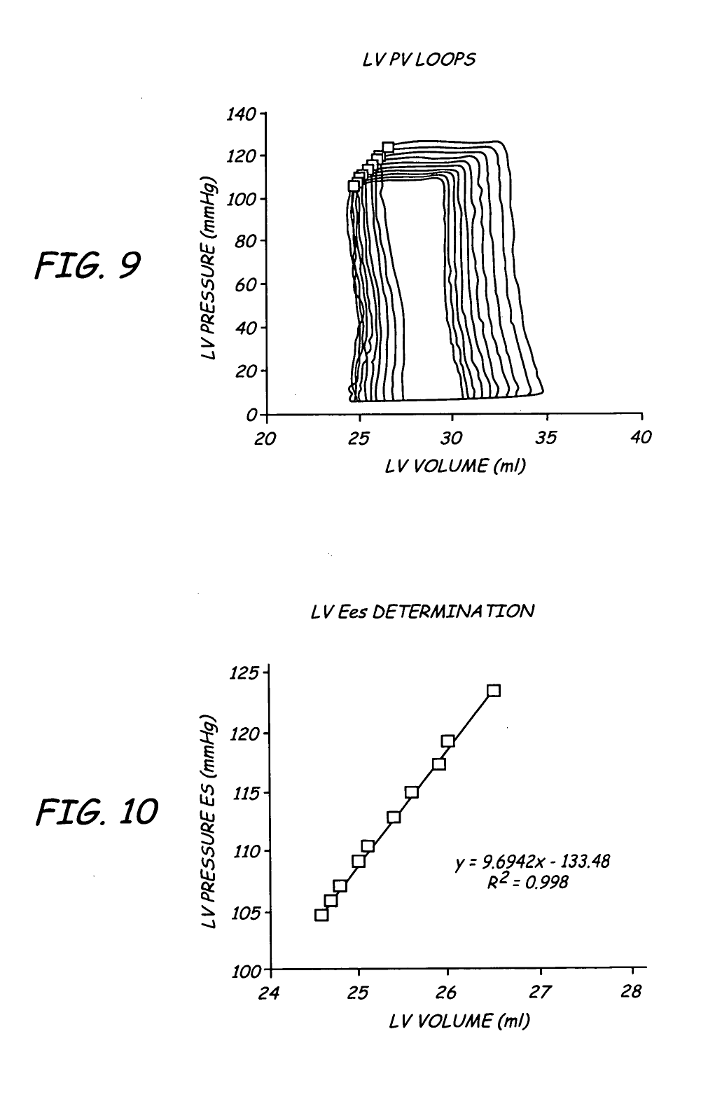

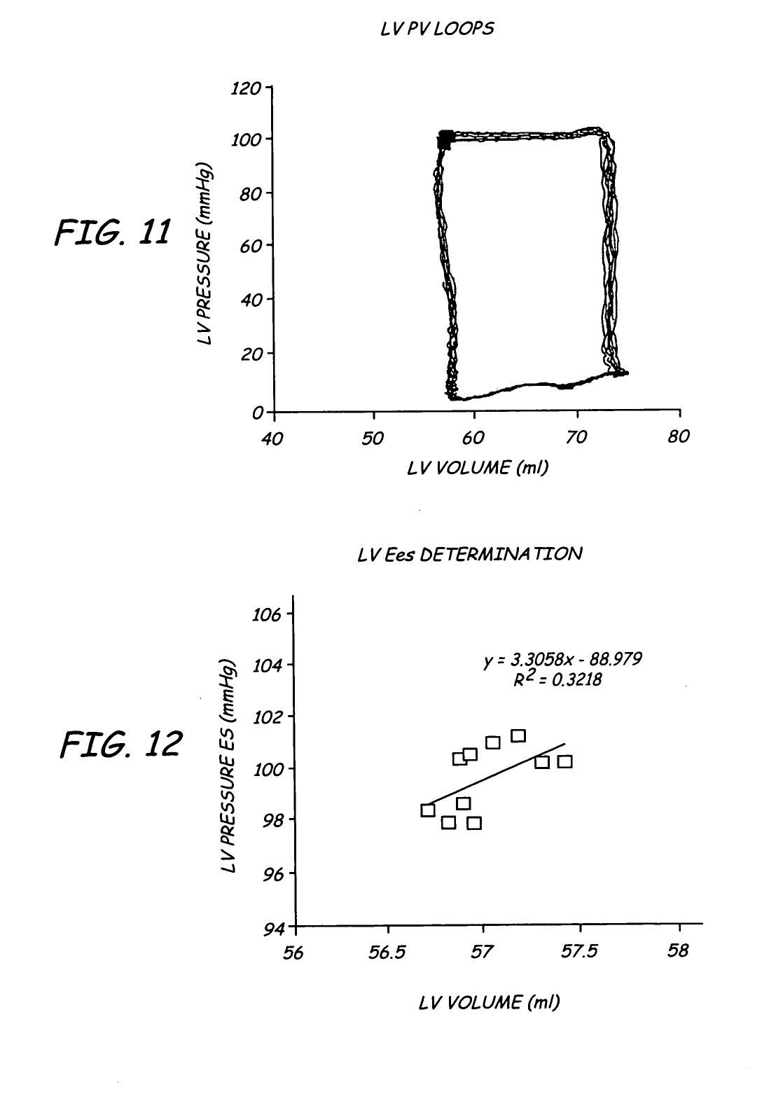

The relationship between pressure and dimension (or volume) provide a closed curve graph when plotted together (as in FIGS. 9 and 11). The dimension measurement during a cardiac cycle has a similar relationship as volume. The width of the closed-loop represents percent of systolic shortening (for dimension) and/or stroke volume (for volume) and the height of the loop represents the developed pressure. The area encircled by the loop is the stroke work. The different phases of the cardiac cycle are also represented in the pressure-dimension/volume relationship loop. The increase in dimension at the bottom of the curve represents filling of the ventricles. The upstroke (and increase in pressure) represents the isovolumetric contraction and the decrease in dimension/volume at the top of the curve represents systole. The downstroke (and decrease in pressure) represents the isovolumetric relaxation of the ventricles and the cycle repeats.

The method and apparatus of the present invention can be provided within a three or four chamber pacing system that can be programmed to restore the depolarization sequence and the synchrony between the right and left heart chambers that contributes to adequate cardiac output. This restoration is effected through providing optimally timed cardiac pace pulses to the RA and/or LA and, after the AV delay, to the RV and LV as necessary and to account for the particular implantation sites of the pace/sense electrodes in relation to each heart chamber while maintaining AV synchrony. The present invention can be employed to obtain data related to the mechanical function of the heart to aid in the assessment of the efficacy of the programmed pacing mode and parameter values and the progression or regression of heart failure.

In accordance with an aspect of the present invention, a method and apparatus is provided to restore the depolarization sequence and the synchrony between the right and left ventricular heart chambers that contributes to adequate cardiac output. This restoration is effected through providing optimally timed cardiac pace pulses to the RA and/or LA and, after the AV delay, to the RV and LV as necessary and to account for the particular implantation sites of the pace/sense electrodes in relation to each heart chamber while maintaining AV synchrony.

Therefore, FIG. 1 also shows a schematic representation of an implanted, four chamber cardiac pacemaker of the above noted types for restoring AV synchronous contractions of the atrial and ventricular chambers and simultaneous or sequential pacing of the right and left ventricles. The pacemaker IPG 14 is implanted subcutaneously in a patient's body between the skin and the ribs. Three endocardial leads 16, 32 and 52 connect the IPG 14 with the RA, the RV and both the LA and the LV, respectively. Each lead has two electrical conductors and at least one pace/sense electrode, and a remote indifferent can electrode 20 is formed as part of the outer surface of the housing of the IPG 14. As described further below, the pace/sense electrodes and the remote indifferent can electrode 20 (IND_CAN electrode) can be selectively employed to provide a number of unipolar pace/sense electrode combinations for pacing and sensing functions, particularly sensing far field signals, e.g. a far field R-wave (FFRS), or bipolar pace/sense electrodes. The depicted positions in or about the right and left heart chambers are also merely exemplary. Moreover other leads and pace/sense electrodes may be used instead of the depicted leads and pace/sense electrodes that are adapted to be placed at electrode sites on or in or relative to the RA, LA, RV and LV.

The depicted bipolar endocardial RA lead 16 is passed through a vein into the RA chamber of the heart 10, and the distal end of the RA lead 16 is attached to the RA wall by an attachment mechanism 17. The bipolar endocardial RA lead 16 is formed with an in-line connector 13 fitting into a bipolar bore of IPG connector block 12. The in-line connector 13 is coupled to an RA lead conductor pair within lead body 15 and connected with distal tip RA pace/sense electrode 19 and proximal ring RA pace/sense electrode 21. Delivery of atrial pace pulses and sensing of atrial sense events is effected between the distal tip RA pace/sense electrode 19 and proximal ring RA pace/sense electrode 21, wherein the proximal ring RA pace/sense electrode 21 functions as an indifferent electrode (IND_RA). Alternatively, a unipolar endocardial RA lead could be substituted for the depicted bipolar endocardial RA lead 16 and be employed with the IND_CAN electrode 20. Or, one of the distal tip RA pace/sense electrode 19 and proximal ring RA pace/sense electrode 21 can be employed with the IND_CAN electrode 20 for unipolar pacing and/or sensing.

Endocardial RV lead 32 is transvenously advanced through the SVC and the IRA and into the RV where its distal tip RV pace/sense electrode 40 is fixed in place in the apex by a conventional distal attachment mechanism 41. In accordance with one aspect of the present invention, a blood pressure sensor 38 and a sonomicrometer crystal 72 are incorporated within a distal segment of the lead body 36 of RV lead 32 to be located within the RV when the distal attachment mechanism 41 attaches to the ventricular apex.

The pressure sensor 38 can be of the type disclosed in the above-referenced '434 patent and employed with the Medtronic® CHRONICLE® IHM monitor. Such implantable monitors when implanted in patients suffering from cardiac arrhythmias or heart failure accumulate date and time stamped data that can be of use in determining the condition of the heart over an extended period of time and while the patient is engaged in daily activities. The conductive surface of the pressure sensor 38 can be employed as an indifferent pace/sense electrode to provide bipolar pacing and sensing with the distal pace/sense electrode 40.

The sonomicrometer crystal 72 can be a cylindrical piezoelectric crystal tube sandwiched between an inner tubular electrode and an outer tubular electrode and fitted around the lead body 36 of the type described in U.S. Pat. No. 5,795,298. Various sonomicrometer systems for measuring distance between an driven piezoelectric crystal acting as a transmitter of ultrasonic energy and a receiving piezoelectric crystal that vibrates when exposed to the ultrasonic energy and provides an output signal are disclosed in U.S. Pat. Nos. 5,779,638, 5,795,298, 5,817,022 and 5,830,144. Cylindrical receiving crystals are mounted to an ECG mapping lead body and coupled to the lead conductors in the '298 patent, and the receiving crystals are employed with externally located transmitting crystals to provide a way to locate the mapping electrodes in the body without use of fluoroscopy.

The outer tubular electrode of the piezoelectric crystal 72 can also be employed as an indifferent pace/sense electrode to provide bipolar pacing and sensing with the distal pace/sense electrode 40.

The RV lead 32 is formed with an RV lead conductor pair within lead body 36 extending from an in-line connector 34 fitting into a bipolar bore of IPG connector block 12. A first conductor or the RV lead conductor pair is connected with distal tip RV pace/sense electrode 40, to the inner tubular conductor of the sonomicrometer crystal 72, and to a first terminal of the pressure transducer 38. A second conductor of the RV lead conductor pair is connected with the outer tubular conductor of the sonomicrometer crystal 72 and to a second terminal of the pressure transducer 38.

In this illustrated embodiment, a multi-polar, endocardial CS lead 52 is advanced through the superior vena cava (SVC), the RA, the ostium of the CS, the CS itself, and into a coronary vein descending from the CS, such as the great vein (GV). The distal pace/sense electrodes 48 and 50 are thus located deep in the GV alongside the LV to allow the depolarization of the LV to be detected and to allow pacing pulses to be delivered to the LV simultaneously with, or in timed relation to the delivery of pacing pulses of the RV. In the illustrated four chamber or channel embodiment, LV CS lead 52 bears proximal LA CS pace/sense electrodes 28 and 30 positioned along the CS lead body 56 to lie in the larger diameter CS adjacent the LA. Typically, LV CS leads and LA CS leads do not employ any fixation mechanism and instead rely on the close confinement within these vessels to maintain the pace/sense electrode or electrodes at a desired site. The LV CS lead 52 is formed with a multiple conductor lead body 56 coupled at the proximal end connector 54 fitting into a bore of IPG connector block 12. A small diameter lead body 56 is selected in order to lodge the distal LV CS pace/sense electrode 50 deeply in a vein branching inferiorly from the great vein GV. It will be understood that LV CS lead 52 could bear a single LA CS pace/sense electrode 28 and/or a single LV CS pace/sense electrode 50 that are paired with the IND_CAN electrode 20 or the ring electrode 21 for pacing and sensing in the LA and LV, respectively.

In accordance with one aspect of the present invention, a sonomicrometer crystal 70 is incorporated within a distal segment of the lead body 56 of LV CS lead 52 to be located alongside the LV at a distance from the sonomicrometer crystal 72. In addition, a sonomicrometer crystal 74 is incorporated within a more proximal segment of the lead body 56 of LV CS lead 52 to be located alongside the LA at a distance from the sonomicrometer crystal 72. The sonomicrometer crystal 74 could alternatively be located more proximally on lead body 56 to locate it in the RA or SVC. Or, an additional sonomicrometer crystal 74 could be located more proximally on lead body 56 to locate it in the RA or SVC or on the RA lead body 15 to locate it in the RA or SVC. The sonomicrometer crystals 70 and 74 can be a cylindrical piezoelectric crystal tube sandwiched between an inner tubular electrode and an outer tubular electrode and fitted around the lead body 36 of the type described in the above-referenced '298 patent. The outer tubular electrodes of the piezoelectric crystals 70 and 74 can also be employed as an indifferent pace/sense electrode to provide bipolar pacing and sensing replacing the indifferent pace/sense electrodes 48 and 28, respectively.

In this case, the CS lead body 56 would encase electrically insulated LV and LA lead conductor pairs extending distally from connector elements of a dual bipolar connector 54. The LA lead conductor pair extends proximally from the more proximal LA CS pace/sense electrodes 28 and 30 and the inner and outer tubular electrodes of the sonomicrometer crystal 74. The LV lead conductor pair extends proximally from the more distal LV CS pace/sense electrodes 48 and 50 and the inner and outer tubular electrodes of the sonomicrometer crystal 70.

The sonomicrometer crystals 70, 72 and 74 are thereby disposed apart and in relation to the LV, RV, and LA. It will be understood that additional or alternative sonomicrometer crystals could be disposed in the RA or SVC. The dimensions D1, D2 and D3 vary during the heart cycle, depending upon the instantaneous state of contraction or relaxation of the heart chambers.

It will also be understood that the IPG 14 can comprise an ICD IPG, and that the one or more or the leads 16, 32 and 52 can also incorporate cardioversion/defibrillation electrodes and lead conductors extending thereto through the lead bodies for delivering atrial and/or ventricular cardioversion/defibrillation shocks in any of the configurations and operating modes known in the art.

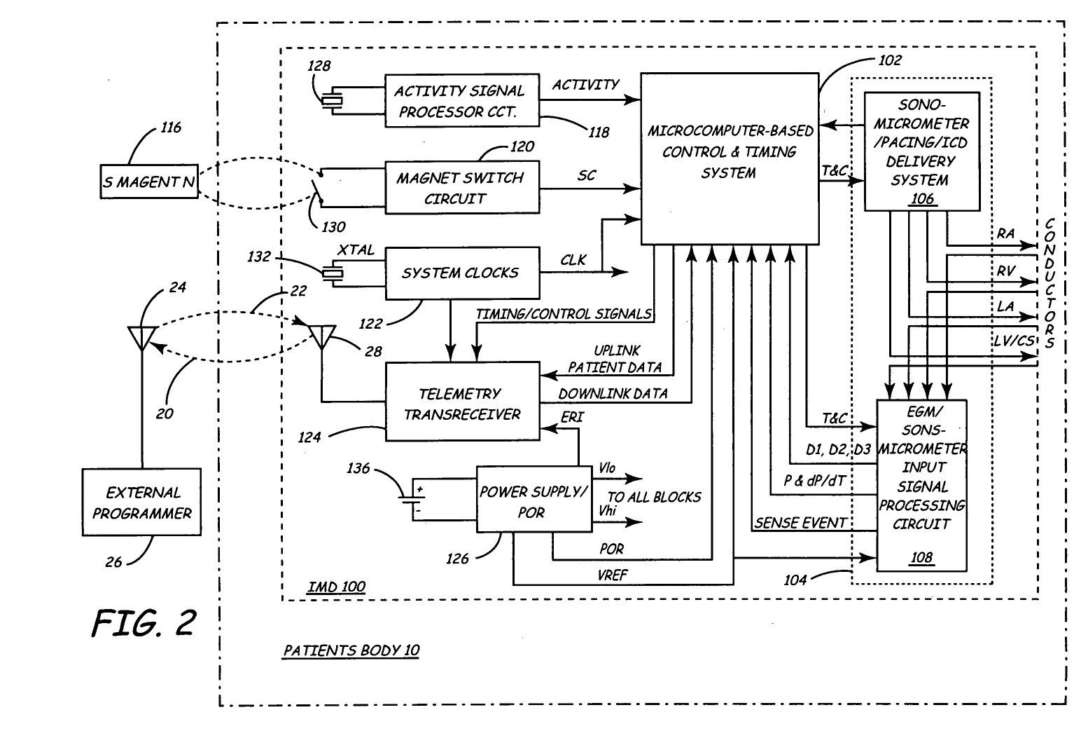

FIG. 2 depicts a system architecture of an exemplary multi-chamber monitor/therapy delivery system IMD 100 implanted into a patient's body 10 that provides delivery of a therapy and/or physiologic input signal processing through the RA, LA, RV and LV lead conductor pairs. The AMD 100 has a system architecture that is constructed about a microcomputer-based control and timing system 102 that varies in sophistication and complexity depending upon the type and functional features incorporated therein. The functions of microcomputer-based multi-chamber monitor/therapy delivery system control and timing system 102 are controlled by firmware and programmed software algorithms stored in RAM and ROM including PROM and EEPROM and are carried out using a CPU, ALU, etc., of a typical microprocessor core architecture. The microcomputer-based multi-chamber monitor/therapy delivery system control and timing system 102 may also include a watchdog circuit, a DMA controller, a block mover/reader, a CRC calculator, and other specific logic circuitry coupled together by on-chip data bus, address bus, power, clock, and control signal lines in paths or trees in a manner well known in the art. It will also be understood that control and timing of multi-chamber IMD 100 can be accomplished with dedicated circuit hardware or state machine logic rather than a programmed micro-computer.

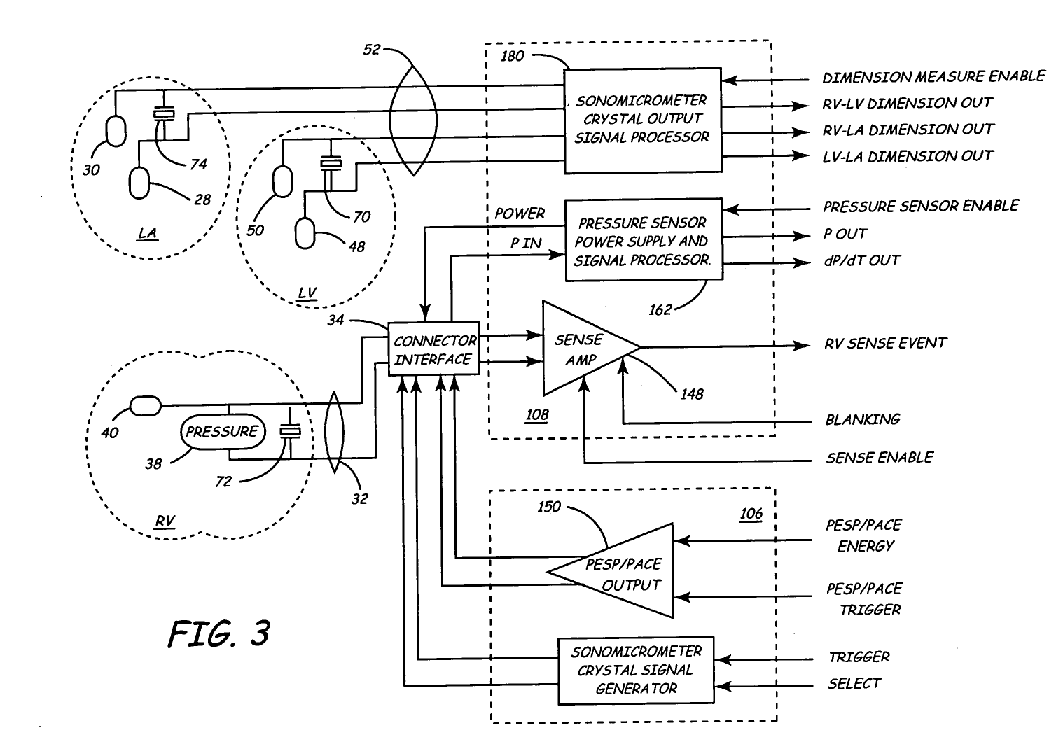

The multi-chamber IMD 100 also typically includes patient interface circuitry 104 for receiving signals from the above-described sensors and pace/sense electrode pairs located at specific sites of the patient's heart chambers to derive heart failure parameters and to time delivery of multi-chamber pacing therapies, particularly AV synchronous, bi-ventricular pacing therapy to the heart chambers. The patient interface circuitry 104 therefore comprises a sonomicrometer/pacing stimulation delivery system 106 and a physiologic input signal processing circuit 108 that are both coupled with the above-described RA. RV, LA and LV lead conductor pairs and described in further detail in reference to FIG. 3. The patient interface circuitry 104 can be configured to include circuitry for delivering cardioversion/defibrillation shocks and/or cardiac pacing pulses delivered to the heart or cardiomyostimulation to a skeletal muscle wrapped about the heart. A drug pump for delivering drugs into the heart to alleviate heart failure or to operate an implantable heart assist device or pump implanted in patients awaiting a heart transplant operation can also be incorporated into the multi-chamber IMD 100.

A battery provides a source of electrical energy to power the multi-chamber IMD 100 and to power any electromechanical devices, e.g., valves, pumps, etc. of a substance delivery multi-chamber monitor/therapy delivery system, or to provide electrical stimulation energy of an ICD shock generator, cardiac pacing pulse generator, or other electrical stimulation generator associated therewith. The typical energy source is a high energy density, low voltage battery 136 coupled with a power supply/POR circuit 126 having power-on-reset (POR) capability. The power supply/POR circuit 126 provides one or more low voltage power sources Vlo, the POR signal, one or more VREF sources, current sources, an elective replacement indicator (ERI) signal, and, in the case of an ICD, high voltage power Vhi to the therapy delivery system 106. Not all of the conventional interconnections of these voltages and signals are shown in FIG. 2.

Virtually all current electronic multi-chamber monitor/therapy delivery system circuitry employs clocked CMOS digital logic ICs that require a clock signal CLK provided by a piezoelectric crystal 132 and system clock 122 coupled thereto as well as discrete components, e.g., inductors, capacitors, transformers, high voltage protection diodes, and the like that are mounted with the ICs to one or more substrate or printed circuit board. In FIG. 2, each CLK signal generated by system clock 122 is routed to all applicable clocked logic via a clock tree. The system clock 122 provides one or more fixed frequency CLK signals that are independent of the battery voltage over an operating battery voltage range for system timing and control functions and in formatting uplink telemetry signal transmissions in the telemetry I/O circuit 124.

RAM memory registers in microcomputer-based control and timing system 102 may be used for storing data compiled from sensed cardiac activity and/or relating to device operating history or sensed physiologic parameters for uplink telemetry transmission on receipt of a retrieval or interrogation instruction via a downlink telemetry transmission. The criteria for triggering data storage can also be programmed in via downlink telemetry transmitted instructions and parameter values The data storage is either triggered on a periodic basis or by detection logic within the physiologic input signal processing circuit 108 upon satisfaction of certain programmed-in event detection criteria. In some cases, the multi-chamber IMD 100 includes a magnetic field sensitive switch 130 that closes in response to a magnetic field, and the closure causes a magnetic switch circuit to issue a switch closed (SC) signal to control and timing system 102 which responds in a magnet mode. For example, the patient may be provided with a magnet 116 that can be applied over the subcutaneously implanted multi-chamber IMD 100 to close switch 130 and prompt the control and timing system to deliver a therapy and/or store physiologic episode data when the patient experiences certain symptoms. In either case, event related data, e.g., the date and time, may be stored along with the stored periodically collected or patient initiated physiologic data for uplink telemetry in a later interrogation session.

Uplink and downlink telemetry capabilities are provided in the multi-chamber IMD 100 to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the multi-chamber IMD 100 to the external programmer or other remote medical device 26 in response to a downlink telemetered interrogation command. The real-time physiologic data typically includes real time sampled signal levels, e.g., intracardiac electrocardiogram amplitude values, and sensor output signals including pressure and dimension signals. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

The physiologic input signal processing circuit 108 includes at least one electrical sense amplifier circuit for amplifying, processing and in some cases detecting sense events from characteristics of the electrical sense signal or pressure sensor output signal. The physiologic input signal processing circuit 108 in multi-chamber monitor/therapy delivery systems providing dual chamber or multi-site or multi-chamber monitoring and/or pacing functions includes a plurality of cardiac signal sense channels for sensing and processing cardiac signals from sense electrodes located in relation to a heart chamber. Each such channel typically includes a sense amplifier circuit for detecting specific cardiac events and an EGM amplifier circuit for providing an EGM signal to the control and timing system 102 for sampling, digitizing and storing or transmitting in an uplink transmission. Atrial and ventricular sense amplifiers include 20 signal processing stages for detecting the occurrence of a P-wave or R-wave, respectively and providing an RA-SENSE. RV-SENSE, LA-SENSE and/or LV-SENSE event signal to the control and timing system 102. Such an RV sense amplifier circuit 48 is depicted in FIG. 3, for example. Timing and control system 102 responds in accordance with its particular operating system to deliver or modify a pacing therapy, if appropriate, or to accumulate data for uplink telemetry transmission or to provide a Marker Channel® signal in a variety of ways known in the art.

FIG. 3 schematically depicts certain of the components of sonomicrometer/pacing stimulation delivery system 106 and input signal processing circuit 108 in relation to the pace/sense electrodes, the pressure sensor 38, and the sonomicrometer crystals 70, 72 and 74 of the LV and RV leads 32 and 52. Not all of the components of the sonomicrometer/pacing stimulation delivery system 106 and input signal processing circuit 108 are depicted in FIG. 3 in order to make its depiction of the components of interest clearer.

The input signal processing circuit 108 includes at least one pressure signal processing channel for sensing and processing pressure sensor derived signals from the RV pressure sensor 38 coupled to the RV lead conductor pair. Such a pressure sensor power supply and signal processor circuit 162 is shown in FIG. 3 coupled to the pressure sensor 38 through connector 34 and the RV lead conductor pair within RV lead body 32.

The sonomicrometer/pacing stimulation delivery system 106 preferably comprises an RA pacing output pulse generator, an RV pacing pulse generator, an LV pacing pulse generator and optionally an LA pacing pulse generator selectively coupled in each case to an RA, RV, LV and LA pace electrode pair which can be programmably selected as described above. For example, the RA pacing output pulse generator can be coupled to the RA lead conductors, the RV pacing pulse generator can be coupled to the RV lead conductors, the LV pacing pulse generator can be coupled to the LV lead conductors, and the LA pacing pulse generator can be coupled to the LA lead conductor pair for bipolar pacing in relation to each chamber. Two, I, m three or four chamber synchronized pacing is effected employing combinations of these pacing pulse generators and following a pacing timing algorithm carried out by microcomputer-based timing and control system 102 in a manner disclosed in commonly assigned, U.S. Pat. No. 5,902,324. PESP pacing pulse trains can also be applied to the selected heart chamber through the selected pace electrode pair in order to increase the force of contraction of the heart during the heart cycle that the paired or coupled stimulation is applied, and the increase persists but gradually diminishes over a number of succeeding heart cycles. The present invention seeks to optimize the timing of delivery of RV and LV pacing pulses to alleviate symptoms of heart failure and optimize cardiac output as a function of measured changes in at least the dimension D2 of FIG. 1.

To this end, FIG. 3 shows that the sonomicrometer/pacing stimulation delivery system 106 comprises a crystal generator 152 for supplying an oscillating drive signal to a programmably selected one of the sonomicrometer crystals 70, 72 and 74 (the driven or ultrasound transmitter crystal). A low energy drive signal at about 1.0 MHz can be applied by crystal generator 152 to the selected one of the sonomicrometer crystals 70, 72 and 74 to transmit the ultrasonic signal through the heart tissue and to induce oscillations at the same frequency in the other selected one or more of the sonomicrometer crystals 70, 72 and 74. In this case, the driven crystal is sonomicrometer crystal 72 coupled through the RV lead conductor pair and lead connector 34 with the crystal generator 152. The transmitted ultrasonic wave energy cause the other sonomicrometer crystals 70 and 74 (in this illustrated case) to vibrate at their resonant frequencies in the manner of a microphone after an RV-LV and RV-LA time delay dependent upon the dimensions DI and D2, respectively, thereby acting as receiver crystals. The ultrasound vibrations develop induced signals that are conducted through the LV and LA lead conductors to and detected by a sonomicrometer signal processor circuit 180 within the input signal processing circuit 108. The RV-LV and RV-LA time delays depend upon the fixed speed of sound through heart tissue, which typically is a constant 1540 meters/second, and the instantaneous distance between the ultrasound transmitter crystal and ultrasound receiver crystal. That distance or dimension varies as a function of how much the LV and LA contracts in the systolic phase and relaxes in the diastolic phase. Sets of instantaneous dimensions DI and D2 can be determined during programmed sample windows of the paced or intrinsic heart cycle from the measured RV-LV and RV-LA time delays collected as the driven sonomicrometer crystal is periodically energized at a defined sample frequency during the defined sample window. The instantaneous LV-LA time delays can also be calculated from the measured RV-LV and RV-LA time delays and employed to determine the instantaneous dimension D3.

Alternatively, the dimensions D1, D2 and D3 can be derived by cycling through a routine of selecting and applying ultrasound energy to RV sonomicrometer crystal 72 and measuring the dimensions DI and D2 as described above and then applying ultrasound energy to LV sonomicrometer crystal 70 or LA sonomicrometer crystal 74 and measuring dimension D3 from the signal received at the other of the LV sonomicrometer crystal 70 or LA sonomicrometer crystal 74. A similar routine may be established if the LA sonomicrometer crystal 74 is located in the RA or SVC.

This determination of the dimensions D1, D2, and D3 compiles accurate data of the excursions of the LV and LA walls due to the locations of the sonomicrometer crystals 70 and 74 without requiring perforation of the LV and LA walls and possible compromise of the functions of the LV and LA.

The RV, LV and LA lead conductors can be employed to power the driven sonomicrometer crystal 72 and to detect the induced ultrasonic frequency signals on sonomicrometer crystals 70 and 74, for example, without compromising the delivery of pacing pulses or the sensing of the atrial and ventricular EGM. The sonomicrometer crystals 70, 72, and 74 exhibit high impedance except at their resonance frequencies of about 1.0 MHz, which is orders of magnitude above pacing pulse and EGM frequency bandwidths. Therefore, the sonomicrometer crystals 70, 72, and 74 act as open circuits and do not conduct or draw current during normal pacing operations but can be periodically energized during sample windows to gather data for storage or adjustment of the AV delay and V-V delay as described further below. The high frequency ultrasound energy is blocked by a filter at the sense amplifier input and protection circuitry at the output of the pacing pulse generators.

Normal Pacing Modes:

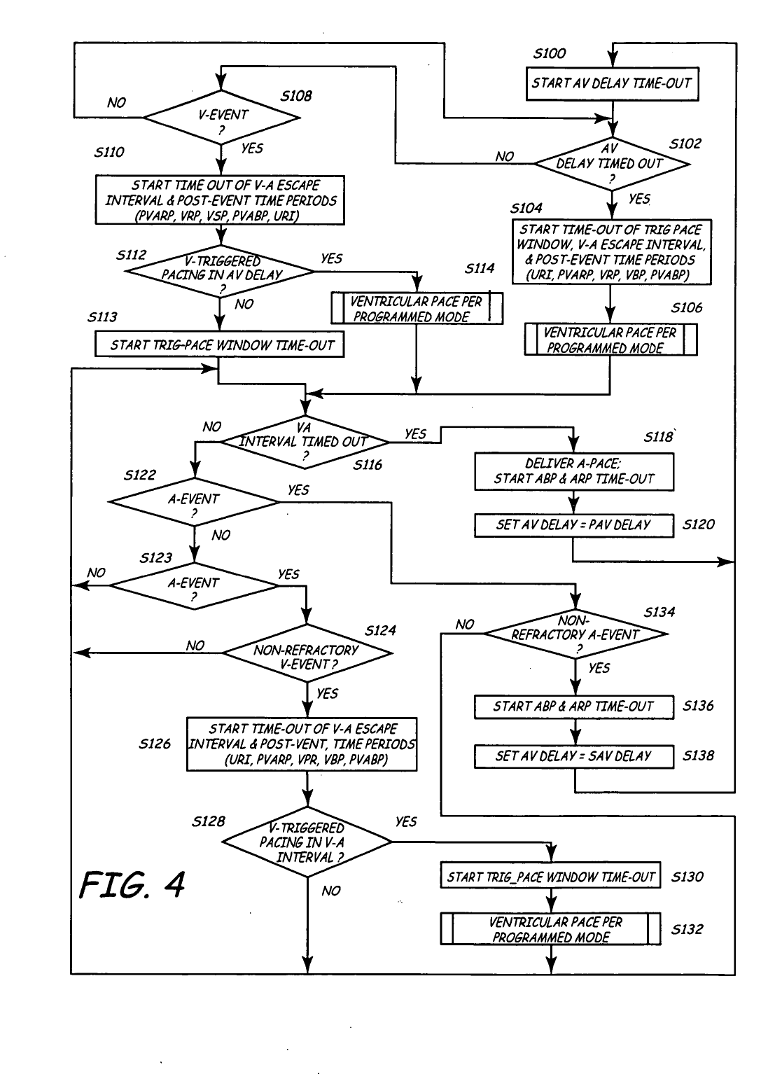

The possible multi-chamber pacing modes of IMD 100 are depicted in the flow chart of FIG. 4 and described as follows. The particular operating modes of the present invention are implemented as a programmed or hard-wired sub-set of the possible operating modes. The AV delay is started in step S100 when a P-wave outside of refractory is sensed across the selected atrial sense electrode pair during the V-A escape interval (an A-EVENT) as determined in step S134 or an A-PACE pulse is delivered to the selected atrial pace electrode pair in step S118. The AV delay can be a PAV or SAV delay, depending upon whether it is started on an A-PACE or an A-EVENT, respectively, and is timed out by the an SAV/PAV delay timer. The SAV or PAV delay is terminated upon a non-refractory RV-EVENT or LV-EVENT output by a ventricular sense amplifier prior to its time-out.

Post-event timers within microcomputer-based control and timing system 102 are started to time out the post-ventricular time periods and the TRIG_PACE window, and a V-A escape interval timer within microcomputer-based control and timing system 102 is started to time out the V-A escape interval in step S104 if the SAV or PAV delay times out in step S102 without the detection of a non-refractory RV-EVENT or LV-EVENT. The TRIG_PACE window inhibits triggered pacing modes in response to a sense event occurring too early in the escape interval.

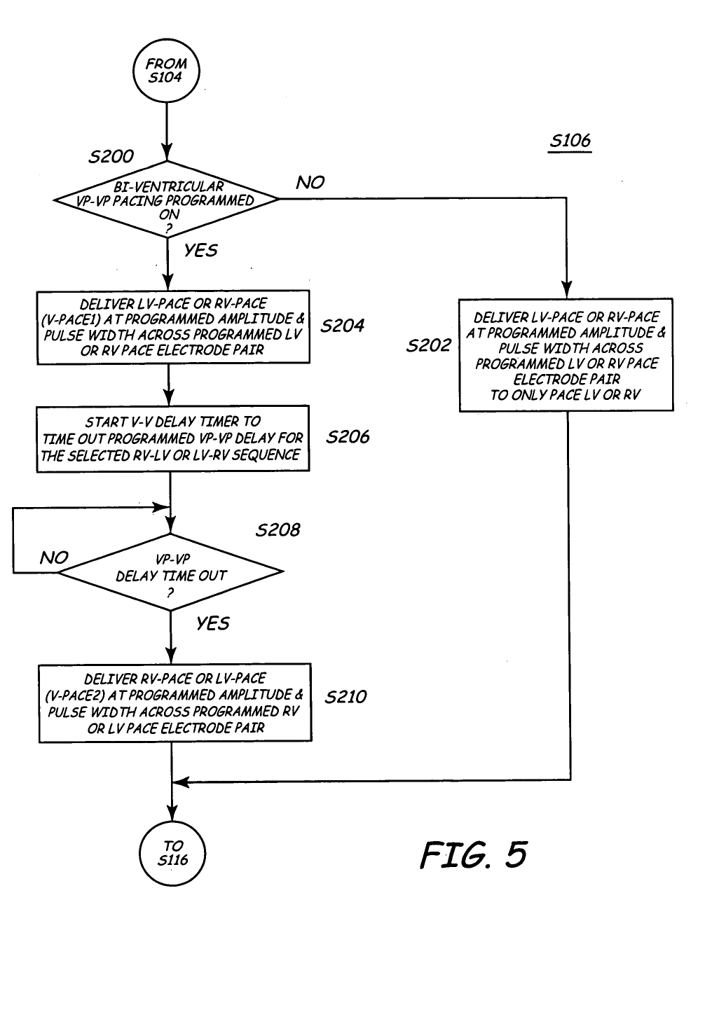

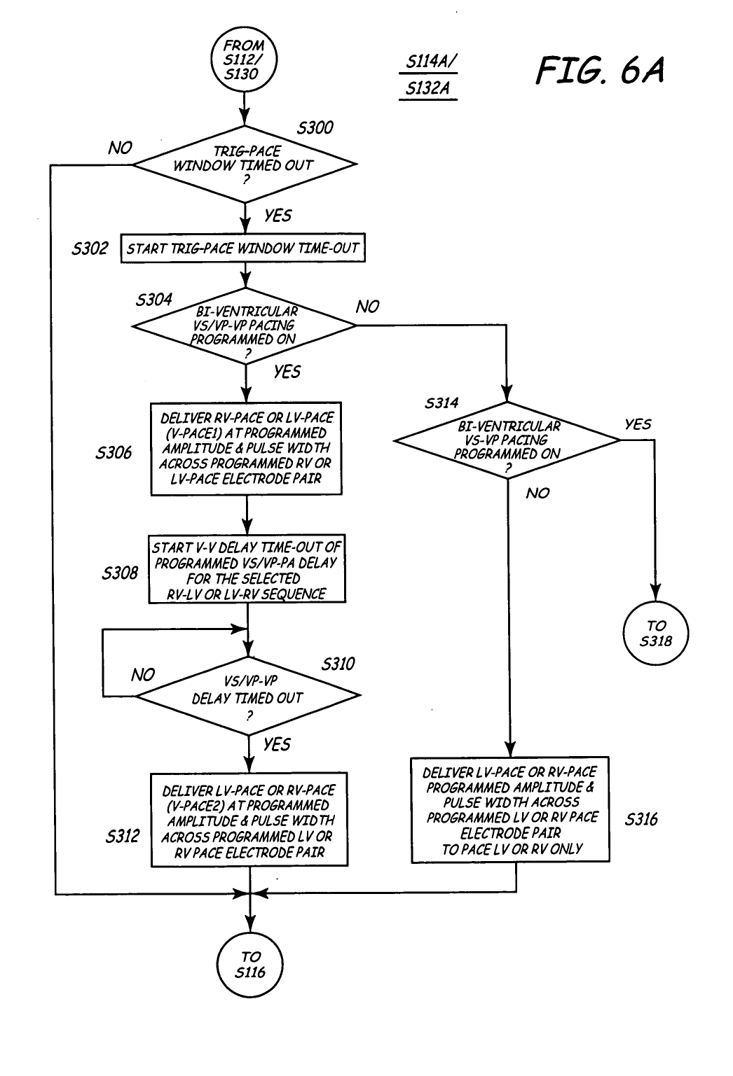

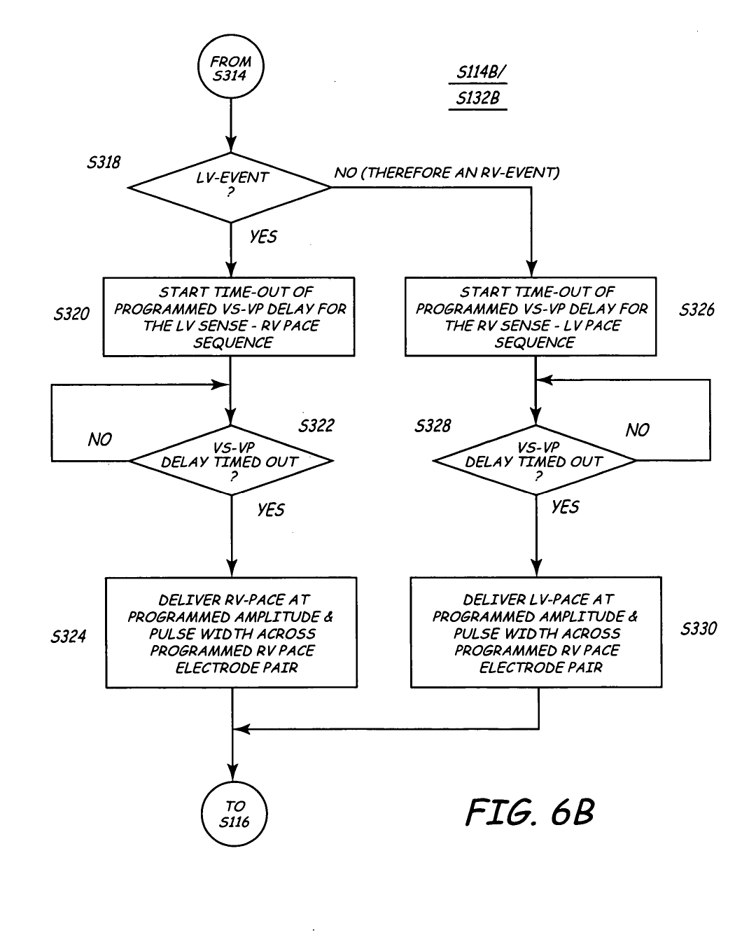

Either a programmed one or both of the RV-PACE and LV-PACE pulses are delivered in step S106 (as shown in the flow chart of FIG. 5) to selected RV and LV pace electrode pairs, and the V-A escape interval timer is timed out in step Si 16. 1: When both of the RV-PACE and LV-PACE pulses are delivered, the first is referred to as V-PACE1, the second is referred to as V-PACE2, and they are separated by a VP-VP delay. As described in greater detail below in reference to FIGS. 6A-6B, if a bi-ventricular pacing mode is programmed in step S106, it can be selectively programmed in a left-to-right or right-to-left ventricle pacing sequence wherein the first and second delivered ventricular pace pulses are separated by separately programmed VP-VP delays. The VP-VP delays are preferably programmable between about 4 msec and about 80 msec.

The baseline or lower rate SAV, PAV and VP-VP delays are initially selected to optimize LA function and LV cardiac output in a patient work-up, typically while the patient is at rest, as described further below. However, these time delays and the V-A escape interval can be programmed to be adjusted within programmed upper and lower limits to accommodate the patient's requirements for cardiac output due to exercise as reflected by the ACTIVITY signal output by the activity signal processor circuit. The pressure (P and dP/dT) and dimension (D1, D2, D3) data associated with the optimum LA function and LV cardiac output are also collected and stored in IMD memory within microcomputer-based control and timing system 102 during the work-up. That data is periodically collected and stored in IMD memory pursuant to the present invention.

Moreover, the pressure (P and dP/dT) and dimension (D1, D2, D3) data can be periodically determined to assess the efficacy of the SAV, PAV and VP-VP delays that are initially selected to optimize LA function and LV cardiac output and to cause the SAV, PAV and VP-VP delays to be adjusted to optimize LA function and LV cardiac output.

Additionally, the pressure (P and dP/dT) and dimension (D1, D2, D3) data can be used to adjust and augment the parameters for delivery of PESP for improving cardiac performance. If necessary, periodic determination of the efficacy of the PESP parameters for improving cardiac function can be performed to maximize performance using the pressure and dimension feedback information for changing PESP parameters.

Returning to step S102, the AV delay is terminated if an RV-EVENT or LV-EVENT (collectively, a V-EVENT) is generated by the RV sense amplifier or the LV sense amplifier in step S108. The time-out of the V-A escape interval and the post-ventricular time periods are started in step S110 in response to the V-EVENT. In step S112, it is determined whether a ventricular triggered pacing mode is programmed to be operative during the AV delay. A ventricular triggered pacing mode is programmed on, and it is undertaken and completed in step S114 (FIGS. 6A-6B). Any VSP mode that may otherwise be available is programmed off. The time-out of the TRIG_PACE window is commenced in step S113 simultaneously with the time-out of the V-A escape interval and post-event time periods in step S110.

The A-PACE pulse is delivered across the selected RA pace electrode pair in step S118, the AV delay is set to PAV in step S120, and the AV delay is commenced by the AV delay timer if the V-A atrial escape interval is timed out in step S116 without a non-refractory A-EVENT being sensed across the selected pair of atrial sense electrodes. But, the V-A escape interval is terminated if a non-refractory A-EVENT is generated as determined in steps S122 and S134. The ABP and ARP are commenced upon an A-EVENT by post-event timers within microcomputer-based control and timing system 102 in step S134, the AV delay is set to the SAV in step S138, and the SAV delay is started in step S100 and timed out by the SAV/PAV delay timer.

Assuming that the normal activation sequence is sought to be restored, a programmed SAV and PAV delay corresponding to a normal AV conduction time from the AV node to the bundle of His are used or a calculated SAV and PAV delay is calculated in relation to the prevailing sensor rate or sensed intrinsic heart rate and are used by SAV/PAV delay timer 372.

If an RV-EVENT or LV-EVENT (for simplicity, referred to as a V-EVENT) is detected in step S123 during the time-out of the V-A escape interval, then, it is determined if it is a non-refractory V-EVENT or a refractory V-EVENT in step S124. If the V-EVENT is determined to be a non-refractory V-EVENT in step S124, then the TRIG_PACE window is started or restarted, the V-A escape interval is restarted, and the post-ventricular time periods are restarted in step S126.

A determination of whether a ventricular triggered pacing mode is programmed to be operative during the V-A escape interval is made in step S128. Ventricular triggered pacing during the V-A escape interval is not programmed on or not provided in the pacing system when triggered ventricular pacing is inappropriate for the patient. If ventricular triggered pacing during the V-A escape interval is programmed on, then it is undertaken and completed in step S132 (FIGS. 6A-6B). If ventricular triggered pacing is not programmed on as determined in step S130, then no ventricular pacing is triggered by the sensed non-refractory V-EVENT during the V-A escape interval. Steps S130 and S132 are merely included herein to complete the disclosure of one form of an AV synchronous pacing system in which the present invention may be incorporated. It will be understood that the present invention can be incorporated into an AV synchronous pacing system that does not include steps S130 and S132.

FIG. 5 depicts the step S106 in greater detail, and FIGS. 6A-6B depict the steps S 114 and S132 in greater detail. If a VP-VP pacing mode is programmed on in step S106, it can be selectively programmed in a left-to-right or right-to-left ventricle sequence, wherein the first and second delivered ventricular pace pulses (V-PACE1 and V-PACE2) are separated by separately programmed VP-VP delays. If a bi-ventricular triggered pacing mode is programmed on in either or both of steps S114 and S132, it can be selectively programmed to immediately pace the ventricle from which the V-EVENT is sensed or a fixed or programmed ventricle regardless of where the V-EVENT is sensed with a V-PACE1. Then, the V-PACE2 is generated to synchronously pace the other ventricle after a programmed VSNVP-VP delay. Or, the triggered pacing mode can be selectively programmed in either or both of steps S114 and 132 to only synchronously pace the other ventricle than the ventricle from which the V-EVENT is sensed with V-PACE2 after separately programmable VS-VP delays, depending on the right-to-left or left-to-right sequence. All of these VP-VP, VS/VP-VP, and VS-VP delays are preferably programmable between nearly 0 msec and about 80 msec.

As a practical matter, the minimum VS/VP-VP, and VP-VP delays may be set to one half the system clock cycle in order to avoid simultaneous delivery of RV-PACE and LV-PACE pulses. The pace pulse width is typically programmable between about 0.5 msec-and 2.0 msec, and the pace pulse amplitude is typically programmable between 0.5 and 7.5 volts. In one embodiment, the system clock provides a full clock cycle of about 8.0 msec. Therefore, the minimum VP-VP delay is set at a half clock cycle or about 4.0 msec.

As shown in FIG. 5, the IMD 100 of FIG. 3 can be programmed to either only deliver a single RV-PACE or LV-PACE (V-PACE1) or the pair of RV-PACE and LV-PACE pulses (V-PACE1 and V-PACE2) separated by the VP-VP delay timed out by a V-V delay timer within microcomputer-based control and timing system 102. If delivery of only a single RV-PACE or LV-PACE is programmed as determined in step S200, then it is delivered in step S202.

If VP-VP pacing is programmed on in step S200, then V-PACE1 is delivered in step S204 in the programmed RV-LV or LV-RV sequence. Again, the RV-PACE pulse is typically delivered across the active RV tip electrode 40 and one of the available indifferent electrodes that is programmed and selected depending upon which are present in the pacing system and the RV pacing vector that is desired as set forth above. And, the LV-PACE pulse is delivered across the active LV pace electrode 50 and a selected indifferent electrode, e.g. pace/sense electrode 48. The V-PACE1 pace pulse is delivered at a programmed pulse energy dictated by the programmed voltage and pulse width.

The V-V delay timer is loaded with the programmed VP-VP delay and starts to time out in step S206. If the RV-PACE pulse is V-PACE1, then a programmed VP-VP delay is timed in V-V delay timer. The LV-PACE pulse is delivered as V-PACE2 in the LV pacing path between the active LV pace/sense electrode 50 and the selected indifferent pace/sense electrode 48 in step S210 after time-out of the programmed VP-VP delay in step S208. Conversely, if the LV-PACE pulse is the first to be delivered (V-PACE1), then a programmed VP-VP delay is timed out in the V-V delay timer. The RV-PACE pulse is then delivered as V-PACE2 typically across the active RV pace/sense electrode 40 and the programmed indifferent pace/sense electrode in step S210 after time-out of the programmed VP-VP delay in step S208.

FIGS. 6A and 6B comprise a flow chart illustrating the steps S114 and S132 (when provided or programmed on) of FIG. 4 for delivering ventricular pace pulses triggered by a ventricular sense event in step S108 during the time-out of an AV delay or in step S124 during time-out of the V-A escape interval. The sensing of R-waves in the RV and LV can be accomplished employing several RV-SENSE and LV-SENSE sensing axes or vectors including a trans-ventricular sensing vector. A bipolar RV-SENSE vector (RV pace/sense electrodes 38 and 40), a unipolar RV-SENSE vector (RV tip pace/sense electrode 40 and IND_CAN electrode 20), and a unipolar LV-SENSE vector (LV pace/sense electrode 50 and IND_CAN electrode 20), and a trans-ventricular, combined RV-SENSE and LV-SENSE vector (RV pace/sense electrode 40 and LV pace/sense electrode 50) can be programmed. The selection of the sensing vectors would depend upon heart condition and the selection of the pace pulse pathways.

The IMD 100 can be separately programmed in one of three triggered pacing modes designated VS/VP, VS/VP-VP or VS-VP triggered modes for step S114. In the VS/VP triggered pacing mode, a V-PACE1 is delivered without delay upon a RV-EVENT or LV-EVENT to the RV or LV pacing pathway, respectively. In the VS/VP-VP triggered pacing mode, the V-PACE1 is delivered without delay upon a RV-EVENT or LV-EVENT to the selected RV or LV pacing electrode pair, respectively, and a V-PACE2 is delivered to the other of the selected LV or RV pacing electrode pair after the VS/VP-VP delay times out. In the VS-VP pacing mode, a RV-EVENT or the LV-EVENT starts time-out of a VS-VP delay, and a single pace pulse (designated V-PACE2) is delivered to the selected LV or the RV pace electrode pair, respectively, when the VS-VP delay times out.

The TRIG_PACE time window started by a prior V-EVENT or V-PACE must have timed out in step S300 prior to delivery of any triggered ventricular pace pulses. If it has not timed out, then triggered pacing cannot be delivered in response to a sensed V-EVENT. If the TRIG_PACE window has timed out, it is then restarted in step S302, and the programmed triggered pacing modes are checked in steps S304 and S316.

When IMD 100 is programmed in the VS/VP-VP triggered mode as determined in step S304, the non-refractory RV-EVENT or LV-EVENT or collective V-EVENT of indeterminable origin is treated as a single V-EVENT. If the TRIG_PACE window has timed out as determined in step S300, then the single V-EVENT triggers the immediate delivery of a programmed one of the RV-PACE or a LV-PACE as V-PACE1 across the programmed bipolar or unipolar RV and LV pace electrode pair, respectively, in step S306. Thus, V-PACE1 is delivered to a predetermined RV or LV pace electrode pair, regardless of whether a RV-EVENT and LV-EVENT is sensed.

Then, a VS/VP-VP delay is started in step S308 and timed out in step S310. The VS/VP-VP delay is specified as a VP-VP delay when the RV-PACE is V-PACE1 and the LV-PACE is V-PACE2. The VS/VP-VP delay is specified as a VP-VP delay when the LV-PACE is V-PACE1 and the RV-PACE is V-PACE2. The LV-PACE or RV-PACE pulse is delivered at the programmed amplitude and pulse width across the programmed LV or RV pace electrode pair in step S210.

In the simplest embodiment of the present invention, the VS/VP-VP mode would be the only triggered ventricular pacing mode provided. The remaining steps of FIGS. 6A and 6B are described in the event that the VS/VP and/or the VS-VP triggered ventricular pacing mode is included in the pacing system.

In step S314, it is determined whether the VS-VP triggered pacing mode or the VS/VP triggered pacing mode is programmed. When the IMD 100 is programmed to a single heart chamber VS/VP triggered pacing mode, the RV-EVENT or LV-EVENT triggers the immediate delivery of an RV-PACE or an LV-PACE across a programmed bipolar or unipolar RV or LV pace electrode pair, respectively, in step S316, regardless of whether an RV-EVENT or LV-EVENT was sensed.

When the IMD 100 is programmed to the VS-VP triggered pacing mode, an LV-EVENT as determined in step S318 loads the appropriate VS-VP delay in V-V delay timer in step S320 and starts the VS-VP delay time-out in step S322. The RV-PACE is delivered at its time-out in step S322 (also designated V-PACE2). If an RV-EVENT is determined in step S318, then the appropriate VS-VP delay in V-V delay timer in step S326 and the VS-VP delay is timed out in step S328. The LV-PACE (also designated V-PACE2) is delivered at time-out of the VS-VP delay in step S330.

In all of steps S306, S312, S316, S324 and S330, the LV-PACE pulse is preferably delivered as V-PACE2 in the LV pacing path between the active LV pace/sense electrode 50 and pace/sense electrode 48.

Returning to FIG. 4, the V-A escape interval is timed out in step Si 16 following the completion of the ventricular pacing mode of FIGS. 6A-6B. If the V-A escape interval times out, then an RA-PACE pulse is typically first delivered across the RA pace electrodes 17 and 19 in step S118, and the AV delay timer is restarted in step S100.

Thus, it will be observed that the multi-site, AV sequential, bi-ventricular cardiac pacing system described above is selectively programmable to provide ventricular pacing pulses delivered to one or both of the RV and LV sites synchronously within a V-V pace delay following time-out of an AV delay from a preceding delivered A-PACE pulse or an A-EVENT (typically, the RA-PACE pulse or the RA-EVENT) and operating in accordance with the steps of: (a) timing an AV delay from a preceding delivered A-PACE pulse or A-EVENT; (b) detecting a V-SENSE at one of a first and second ventricular site within the AV delay and, in response, terminating the AV delay and providing a V-EVENT; (c) delivering a V-PACE1 pulse to a selected one of the first and second ventricular sites upon the time-out of the AV delay or, in a triggered mode, upon the V-SENSE; (d) timing a V-V pace delay comprising one of a VS-VP pace delay from a V-EVENT occurring prior to the time-out of the AV delay or a VP-VP pace delay from the V-PACE1 delivered at the end of the AV delay or a VSNVP-VP pace delay from a triggered V-PACE1; and (e) delivering a V-PACE2 pulse to the other of the first and second ventricular sites upon the time-out of the V-V pace delay.

Mechanical Heart Function Measurement and Optimization:

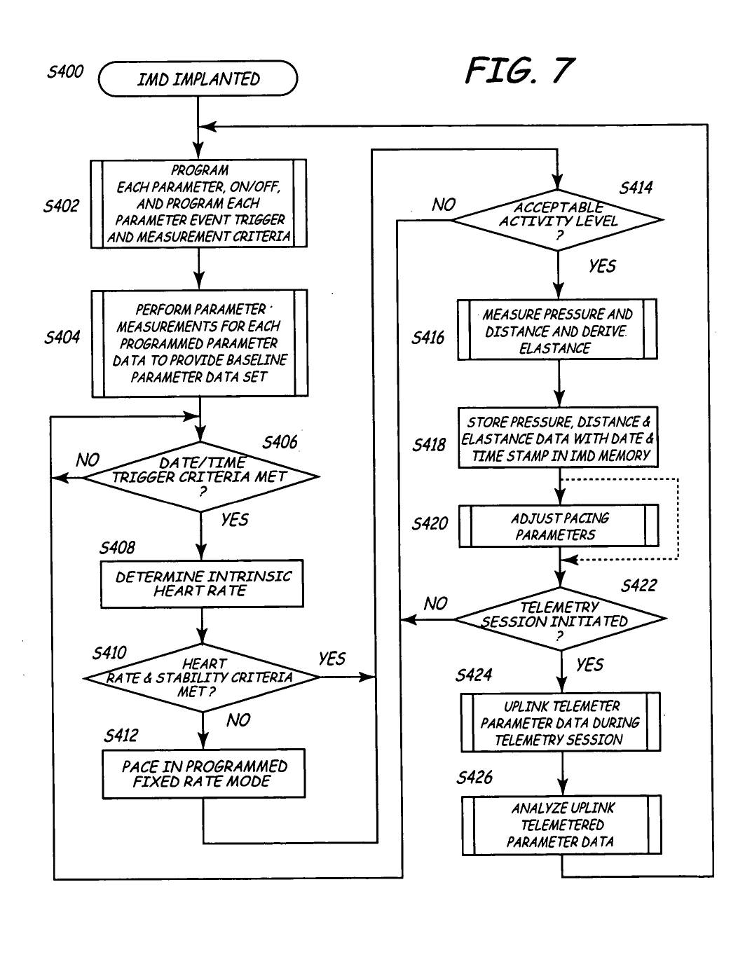

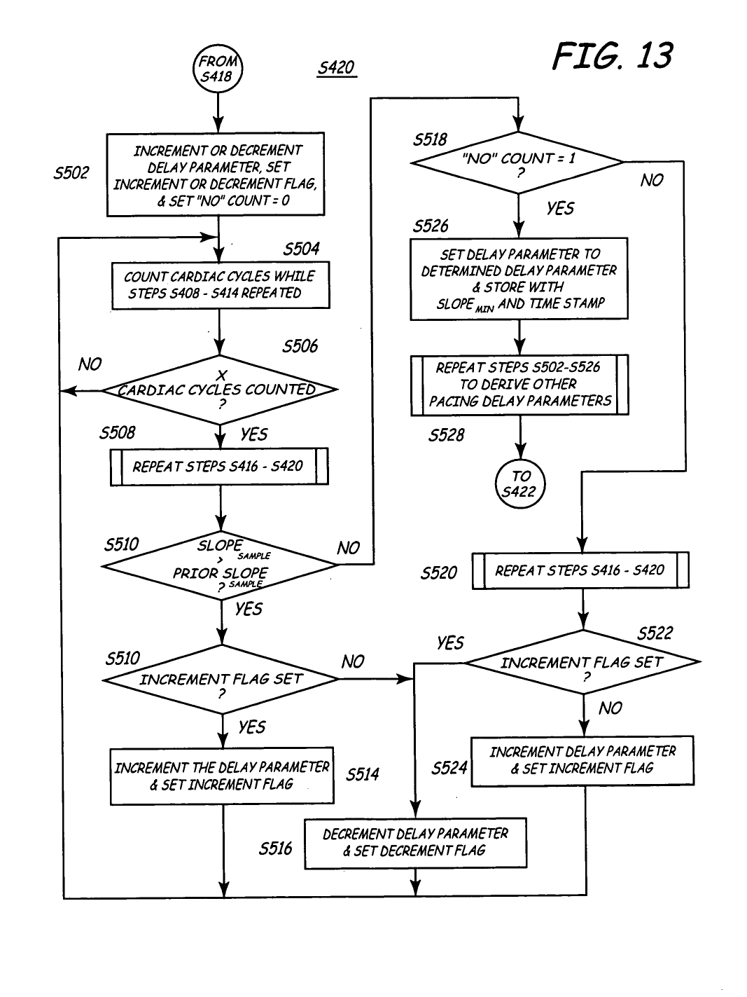

FIG. 7 illustrates the overall IMD function from the time of implantation (step S400) and initial programming (steps 402) and baseline parameter measurements (step S404) through successive cycles of gathering parameter data in the IMD (steps S406-S418), optionally adjusting pacing parameters in step S420 (further described in reference to FIG. 13), uplink telemetry transmission of the accumulated data to an external programmer (step S424) for display and analysis (step S426), leading to possible reprogramming (step S402) and baseline parameter measurement (step S404) to better assess the heart failure state. The present invention may be implemented into a versatile multi-chamber pacing system as described above or into a less comprehensive pacing system offering fewer programmable pacing parameters and operating modes.

Each measured parameter may be programmed ON or OFF, and a particular event trigger for starting measurement of the programmed ON parameter as well as any specific measurement criteria can be programmed in step S402 using conventional downlink telemetry transmitted commands that are received in the telemetry transceiver 124 and forwarded to the microcomputer-based control and timing system 102. The physician may initially program the pacing system to deliver a pacing therapy in accordance with options provided in the flow charts of FIGS. 4, 5 and 6A-6B as described above. At a minimum, the pacing system of IMD 100 would be programmed to operate as a bi-ventricular pacing system or as an AV synchronous bi-ventricular pacing system.

In step S404, baseline parameter measurements are optionally performed for each programmed ON parameter to collect baseline or reference parameter data, to both store such data in IMD memory and to uplink telemeter the parameter data for observation by the physician and for use in programming the operating modes and parameter values. The initial and updated baseline parameter measurements can be stored in the IMD RAM memory and/or stored externally in a patient file maintained by the physician with a date and time stamp and other pertinent data, e.g. patient activity level measured by activity signal processor circuit 118 and patient heart rate, if measurable.

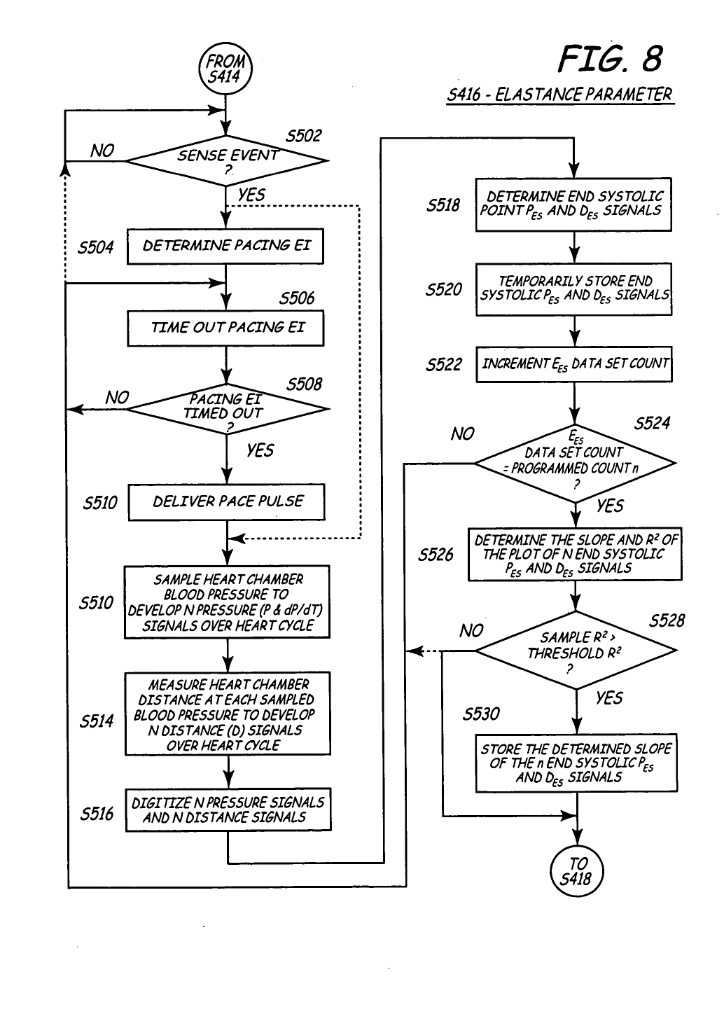

In accordance with the present invention, the RV and/or LV pressure P and dP/dt signals and the dimension data (D1 or D1, D2 and optionally D3) are derived by activating the system depicted in FIG. 3 for each of a plurality of programmed AV delays and V-V delays. Parameter values are derived by following the processes illustrated in FIGS. 7 and 8 and described further below.

In addition, particular selected ones of V-V conduction times (including the VP-VS and/or VP/VS-VS and/or VS-VS conduction times) can be collected from a paced or sensed ventricular event, (typically the RV-PACE or RV-EVENT to the LV-EVENT). If AV sequential pacing is operative, then the PAV and SAV delays from a paced or sensed atrial event (typically the RA-PACE or RA-EVENT) to a V-EVENT (typically the first to occur of the RV-EVENT and the LV-EVENT) are also collected. Other data, e.g. the RV and LV QRS duration signals can also be collected and employed in at least initially optimizing the cardiac output.

After implant, the programmed ON parameters are measured in step S416 when an event trigger for the specific parameter occurs and when heart rate and/or rhythm criteria and patient activity level criteria are met as set forth in steps S408-S414. The event criteria of step S406 may be a programmed time or multiple times of every day or specified days of the week or month as tracked by a date/time clock within the microcomputer-based timing and control system 102 or the detection of the patient initiated parameter measurement or some other programmed event, e.g., a combination of the time or times of day and a level of patient exercise indicated by the activity signal processor circuit 118.

Typically, the collection of the data in step S404 and step S416 should take place when the heart rate is in a normal range and is stable within a certain stability tolerance which can both be programmed by the physician and are determined over a series of heart cycles in steps S408-S412 in a manner well known in the art. The measurement of the data also only takes place in step S416 when the patient activity level is appropriate, e.g., reflecting rest or steady activity, as determined in step S414. Typically, in step S408, incidences of spontaneous RA-EVENTs and RV-EVENTs would be monitored while the escape interval establishing the pacing rate is set to the lower rate limit (LRL) to determine the intrinsic heart rate.

The heart rate would be established at the pacing LRL or another programmed rate in step S412 if the intrinsic heart rate cannot be determined in this way or is unstable as determined in step S410. The atrial and ventricular pacing pulses will be delivered during the test if the patient's intrinsic heart rate is lower than the LRI established pacing rate, and consequently the heart rate will be inherently low and stable under these circumstances.

The measurement and storage of the particular pressure and dimension data is then conducted in step S416 over a programmed number of heart cycles or a time period if the activity level criteria are met in step S414. The heart rate and/or stability continues to be monitored through steps S416-S420, and the pressure and dimension measurement that is commenced in step S416 may also be aborted if the heart rate and/or stability changes such that the heart rate/stability criteria become no longer satisfied in step S410 before the parameter measurement steps are completed.

The physician may program the IMD 100 to perform one or more of the pressure and dimension measurements in a single session initiated in step S406. In each case, a single pressure and dimension value can be obtained and stored in steps S416 and S418 or the maximum, minimum and average pressure and dimension values can be obtained in step S416 and stored in IMD memory with a date and time stamp and any other pertinent information, e.g., patient activity level, in step S418. The history of the number, times and dates of successive parameter measurements can also be stored in IMD memory, but the stored parameter data and related data may be discarded on a FIFO basis if the memory capacity assigned to such data storage is exceeded.

Steps S408 through S418 are repeated each time that the event trigger criteria for the V-V conduction time measurement are satisfied in step S406. The data collection continues until the accumulated data is uplink telemetered to the physician in steps S422 and S424. The physician then reviews the accumulated data in step S426 to determine if the pressure and dimension data reveals a trend. Pressure and dimension trend data evidencing any change in the intrinsic or triggered V-V conduction time between RV and LV sites gathered over a period of days, weeks and months provides a valuable indication as to whether the heart failure state is improving, worsening or staying about the same. The physician can then reprogram pacing operating modes and parameter values in steps S402 and S404 to provide a more efficacious therapy.

In addition, the IMD can be programmed to perform step S420 as depicted in FIG. 13 to optimize pacing parameter values when the criteria of steps S406-S414 are satisfied.

The preceding specific embodiments are directed AV sequential pacing wherein typically the atrial pacing and sensing takes place in one of the RA and LA and ventricular pacing takes place in a predetermined one of the RV-LV or LV-RV sequence at ventricular sites in the RV and LV. However, it will be understood that the present invention also embraces locating first and second ventricular pace/sense electrodes separated apart from one another but within either the RV or LV.

Collection of End Systolic Elastance Parameter Data:

The raw collected pressure and dimension trend data may be of use in monitoring the state or progression of heart failure. Moreover, the end systolic elastance E_{ES} parameter is believed to be a useful indicator of the state of heart failure and can provide an indication of the state of progression or regression of the heart failure through the comparison of E_{ES} parameter data collected over time. The end systolic elastance E_{ES} parameter comprises a slope determined from a collection or “cloud” of “n” data points of end systolic P_{ES} measurements plotted against the simultaneously determined end systolic heart chamber volume DES measurements.

FIG. 8 depicts the steps of determining the E_{ES} parameter in step S416 of FIG. 7. When the E_{ES} parameter measurement is started, it can be conducted during “n” successive paced heart cycles as illustrated in steps S504-S506 or during intrinsic heart cycles as illustrated by the broken lines. In the latter case, it may be advisable to make a determination that the heart rate and rhythm remain within prescribed ranges between steps S502 and S512. In the former case, the pacing Escape Interval (El) is calculated that is sufficiently shorter than the intrinsic EI to overdrive pace the heart chamber in step S504, and fixed rate pacing is carried out in steps S504-S508 at least for “n” programmed pacing cycles.

In either case, the pressure sensor power supply and signal processor 162 is enabled in step S512 to measure the heart chamber blood pressure and provide “N” sampled P and dP/dt signals over the heart cycle. At the same time, the sonomicrometer crystal signal generator 152 is enabled in step S514 to develop “N” dimension [D1, D2, D3] signals over the heart cycle. The “N” sampled P and dP/dt and dimension [D1, D2, D3] signals are digitized in step S516 and applied to control and timing system 102.

The end systolic point P_{ES} and D_{ES} is determined in step S518 and stored in IMD memory in step S520. The determination of the end systolic P_{ES} and D_{ES} samples at the end systolic point in the heart cycle is made by first determining dP/dt MIN sample and selecting a P sample and D1 sample at a short time, e.g., 20 ms, prior to the dP/dt MIN sample. In this way, “n” sets of [P_{ES}, D_{ES}] data points are accumulated for determination of E_{ES} and derivation of a correlation coefficient R and squared correlation coefficient R² in step S526.

The E_{ES} data set count is then incremented in step S522, and the incremented count is compared to a programmed data set count “n” in step S524. The process of determining the n end systolic point P_{ES} and D_{ES} values is commenced again for the next intrinsic El at step S502 or the next paced El at step S504, and the process is repeated until the programmed data set count “n” is reached.

It should also be noted that the event trigger criteria of step S406 can be programmed in step S402 to be “all times” that step S412 is met or fixed rate pacing is provided in steps S504-S508. In this case, “n” sets of [P_{ES}, D_{ES}] data points are continuously accumulated on a FIFO basis for determination of E_{ES} and derivation of a correlation coefficient R and squared correlation coefficient R² in step S526. In this variation, steps S522 and S524 are always satisfied when the first “n” sets of [P_{ES}, D_{ES}] data points are accumulated.

Then, in either case, in step S526, a linear regression of the “n” sets of [P_{ES}, D_{ES}] data points is conducted using standard linear regression techniques to derive the slope of the sampled data set, E_{ES}, a correlation coefficient, R, and the squared correlation coefficient R² as depicted in FIGS. 9-11 as described further below.

In step S528, the squared correlation coefficient R² of the “n” sets of [P_{ES}, D_{ES}] data points data set (the sample squared correlation coefficient R²) is compared to a threshold squared correlation coefficient R² (e.g. 08-0.9) that is initially programmed in step S402.

The slope of the sampled data set of “n” end systolic [P_{ES}:V_{ES}] data points determined in step S526 is saved as the E_{ES} in step S530 if the sample squared correlation coefficient R² exceeds the threshold squared correlation coefficient R² value as determined in step S528. If the threshold condition is not met, then a slope of the sampled set of “n” end systolic [P_{ES}, D_{ES}] values cannot be meaningfully determined. The accumulated data set is either discarded and the E_{ES} parameter measurement aborted as shown in FIG. 7 or the data set is updated on a FIFO basis by starting again at either step S502 or step S506. The accumulated data set and/or slope E_{ES} is then saved with other associated data in IMD memory in step S530 if the slope can be determined from the clustered plotted intersecting data points of “n” end systolic [P_{ES}, D_{ES}] values.

Dimension and volume follow the same relationship with respect to pressure for the pressure-volume relationship during the cardiac cycle. Dimension is reduced during systole similar to a reduction in ventricular volume during systole and likewise an increase in dimension during ventricular filling similar to an increase in ventricular volume during filling. Multiple dimensions can be used to estimate volume similar to the volumetric measures used in echocardiography for estimates of ventricular volume from two-dimensional measurements.

FIG. 9 is a plot of ten consecutive PD loops during a modification of preload (vena caval partial occlusion) with end systolic PD points shown at the upper left of FIG. 9. When a linear regression is performed using these ten end systolic PD points of FIG. 9, a straight line is formed as shown in FIG. 10. The fit of the line shown in FIG. 10 to the systolic PD points is very good with correlation R²=0.998. An end systolic elastance E_{ES} of 9.69 is evidenced by the slope of the line. It is expected that the slope will change in a manner that signifies the progression or remission of heart failure in a patient's heart.

By contrast, FIG. 11 is a plot of ten consecutive PD loops at a baseline condition of a relatively normal heart evidencing little physiologic change in the measured P and D. As a result, the ten end systolic PD points are on top of each other in the upper left corner of FIG. 11. When a linear regression is performed using these ten end systolic PD points in FIG. 12, these points do not reliably form a good straight line and thus do not permit an estimation of E_{ES}. The correlation of R²=0.322 is sufficient to recognize that the E_{ES} slope of 3.31 is not an accurate reflection of the physiology and would be discarded following the comparison step S526.

The end systolic elastance E_{ES} is computed periodically or continuously in this manner to store a set of such slopes. The stored slopes are retrieved by uplink telemetry to an external programmer and are subjected to linear regression analysis to determine if a more recent slope has changed from an earlier slope in a manner that signifies a deterioration or improvement in CHF. A decrease in E_{ES} implies a decrease in systolic function and loss in contractile strength.

It will be appreciated from the above description that the implanted monitor/stimulator of the present invention may be utilized to obtain the aforementioned parameters as stored patient data over a period of time. The treating physician is able to initiate uplink telemetry of the patient data in order to review it to make an assessment of the heart failure state of the patient's heart. The physician can then determine whether a particular therapy is appropriate, prescribe the therapy for a period of time while again accumulating the stored patient data for a later review and assessment to determine whether the applied therapy is beneficial or not, thereby enabling periodic changes in therapy, if appropriate. Such therapies include drug therapies and electrical stimulation therapies, including PESP or other bust stimulation therapies, and pacing therapies including single chamber, dual chamber and multi-chamber (bi-atrial and/or bi-ventricular) pacing. Moreover, in patients prone to malignant tachyarrhythmias, the assessment of heart failure state can be taken into account in setting parameters of detection or classification of tachyarrhythmias and the therapies that are delivered.

It would be desirable to employ the pressure and dimension data and E_{ES} elastance to derive the AV delay and V-V pace delay or other parameters. e.g., the parameters of burst stimulation therapies, that optimizes cardiac output as measured by the elastance E_{ES}. FIG. 13 is a flow chart illustrating step S420 in deriving a set of pacing parameters providing optimal right and left mechanical heart function that are employed until step S416 is repeated.

In step S420, incremental changes are automatically made to the SAV delay, PAV delay and/or V-V delay, and the effects of the changes as evidenced by changes in the slope of the E_{ES} derived in a series of P and D measurements made after each change are determined as would be done in the external programmer as described above. Step S420 can be programmed on or off and thereby bypassed in FIG. 7. No parameter changes are made if step S420 is programmed off, but the physician still obtains valuable data illustrating the trend in elastance E_{ES} in the course of following the steps of FIG. 7 that can be analyzed to determine whether the patient's heart failure state is improving or deteriorating. If it appears that the elastance E_{ES} is remaining stable or increases over time, then it may be presumed that the applied pacing therapy and drug therapy is of benefit. If the elastance E_{ES} is decreased, then adjustments in therapy, including repeating steps S402 and S404 need to be undertaken.

In one variation of this aspect of the invention, the delay parameters comprising one or more of the LRL, the SAV delay, the PAV delay, the A-A delay, and/or V-V delay, providing the optimal elastance E_{ES} is derived. The selected delay parameter is successively incremented or decremented, an elastance E_{ES} value at each adjusted delay is derived and compared to the preceding derived elastance E_{ES} value to determine if the elastance E_{ES} value is increased or decreased. The delay parameter is setting to the newly derived delay parameter value that provides the optimal elastance E_{ES} value.

One manner of determining the values of the LRL, SAV delay, PAV delay, A-A delay and/or V-V delay that provide the optimal elastance E_{ES} value is illustrated in FIG. 13. An alternative to this dithering approach is to have a preset threshold or boundary of the value. If the observed value exceeds the threshold or extends beyond the boundary limits, then the algorithm is engaged.

In step S418, the first measured elastance E_{ES} value at the prevailing LRL, A-A delay, V-V delay, SAV delay and PAV delay has been stored in step S418. A point-in-time measurement of elastance assumes that the unstressed volume of the ventricle remains stable over the test/measurement period.

Each of a series of elastance E_{ES}_{_{—}}_{SAMPLE} values that are measured after a change in one or more of the LRL, A-A delay, V-V delay, SAV delay, and PAV delay are compared with the preceding or prior measured elastance E_{ES}_{_{—}}_{SAMPLE} value to determine if the change has increased the slope. An additional change in the same direction (increasing or decreasing the parameter duration) is made if the prior change increases the slope. But, if the change results in a decreased slope, then the change direction is reversed to repeat the measurement of the elastance E_{ES} using the prior parameter value. Only one reversal in direction is allowed to inhibit “hunting” that could otherwise occur and cause the algorithm to repeat the dithering indefinitely. A rest period of a number of heart cycles or a time period is provided between each change in a LRL, A-A delay, V-V delay, SAV delay, and PAV delay parameter value to allow the heart to acclimate to the change.

Thus, in step S502 one or more of the LRL and/or SAV delay and/or PAV delay and/or A-A delay and/or V-V delay are either incremented or decremented, the corresponding increment or decrement flag is set so that the direction of change (increase or decrease) is recorded, and a “NO” count is set to “0”. Then, the resting period is timed or counted out in steps S504 and S506. It will be understood that a physician may establish an incrementing and decrementing routine from the patient work-up in steps S402 and S404 to determine which of the parameters and combinations of parameters effect a change in the elastance E_{ES} in the particular patient. The physician can also program the increment and decrement amounts and the length of the resting period of steps S504 and S506. The physician can also program the system to abort or continue the process after a delay if steps S410 or S414 are not satisfied.

At this point, steps S416-S418 are repeated per step S508 to derive a succeeding measured E_{ES}_{_{—}}_{SAMPLE} value at the decremented or incremented one or more of the LRL, A-A delay, V-V delay and/or SAV delay and/or PAV delay that is can be stored in memory in step S418 to retain a record of the operation of the algorithm for retrieval and review by the physician in a subsequently initiated telemetry session. The succeeding measured E_{ES}_{_{—}}_{SAMPLE} value is compared to the prior measured E_{ES}_{_{—}}_{SAMPLE} value in step S510. If the succeeding measured E_{ES}_{_{—}}_{SAMPLE} value is greater than the prior measured E_{ES}_{_{—}}_{SAMPLE} value, then the flag status is checked in step S512. If the increment flag was set in step S502, and the increment has effected the favorable increase in the elastance E_{ES}, then the one or more of the SAV delay and/or PAV delay and/or V-V delay that was incremented in step S502 is again incremented in step S514. Similarly, if the decrement flag was set in step S502, and the decrement has effected the favorable increase in the elastance E_{ES}, then the one or more of the LRL, SAV delay and/or PAV delay, A-A delay and/or V-V delay that was decremented in step S502 is again decremented in step S516. The process of steps S504-S516 is then repeated to determine if the increase in the elastance E_{ES} can be further increased.

Returning to step S510, if the succeeding measured elastance E_{ES}_{_{—}}_{SAMPLE} value is greater than the prior measured E_{ES}_{_{—}}_{SAMPLE} value, which can occur in the first pass through steps S502 through S508 or in subsequent passes through S504-S516, then a change in direction is initiated. The “NO” count (set to “0” in step S502) is checked in step S518 and incremented to “1” in step S520. The flag status is checked in step S522 to determine the prevailing direction of change, and the change in direction is effected in step S516 or S524. Thus, if the one or more of the LRL, A-A delay, SAV delay and/or PAV delay and/or V-V delay was decremented previously, then the direction is changed in step S524 to increment the one or more of the LRL, A-A delay, SAV delay and/or PAV delay and/or V-V delay and to repeat steps S504-S510.

At some point, the succeeding measured E_{ES}_{_{—}}_{SAMPLE} value is greater than the prior measured E_{ES}_{_{—}}_{SAMPLE} value, and the condition of step S518 is satisfied. Then, the prior measured E_{ES}_{_{—}}_{SAMPLE} value is declared the optimal elastance E_{ES}, and it and the corresponding one or more of the LRL, A-A delay, SAV delay and/or PAV delay and/or V-V delay are stored in RAM and employed in the operating system as described above with respect to FIGS. 4 through 6B until step S420 is repeated upon a trigger event satisfying step S406 and satisfaction of the criteria or steps S408-S414.

Alternatively, the incremented or decremented preceding value of the one or more of the LRL, A-A delay, SAV delay and/or PAV delay and/or V-V delay are stored in RAM and employed in the operating system as described above with respect to FIGS. 4 through 6B the first time the condition of step S510 is not satisfied.

The physician can also enter programming commands that enable successive changes in each of the pacing parameter values including the LRL, A-A delay SAV delay, PAV delay and V-V delay to be tested pursuant to steps S502-S526 and the above-described variants. Therefore, the next one of the synchronous pacing delays can be tested after a previous synchronous pacing delay has been derived by repeating steps S502-S526 pursuant to step S28 until all of the delay values have been derived. In many clinical cases, only the optimal V-V delay in the RV-LV or LV-RV sequence would be obtained. In other clinical cases, the optimal SAV delay would be first obtained, and then the optimal V-V delay in the RV-LV or LV-RV sequence would be obtained. In certain clinical cases, the PAV delay would be automatically set to be the same as the optimal SAV delay derived through steps S502-S526. The order of the process and the tests included in the process can be left to the clinicians to develop for the particular patient.

The resulting pacing parameter values of the LRL, SAV delay, PAV delay, A-A delay and/or the V-V delay are stored with the corresponding elastance E_{ES} data and the other related data in step S526 and employed in the operating system depicted in FIGS. 4 through 6B until the event criteria are next satisfied. Therefore, in this aspect, the present invention can be employed to selectively derive the LRL and/or SAV delay and/or PAV delay and/or A-A delay and/or the V-V delay that optimizes the elastance E_{ES} over a period of weeks or months until the physician is able to analyze the stored data in step S428 and perform steps S402 and S404 if deemed desirable.

A similar algorithm to that depicted in FIG. 13 can be employed to derive the optimal parameters of PESP or other burst stimulation therapies for delivery to the patient. In this variation, the burst stimulation therapy parameters can be altered instead of the LRL, SAV, PAV, A-A delays and V-V delays in steps S502, S514, S516, and S524-S528.

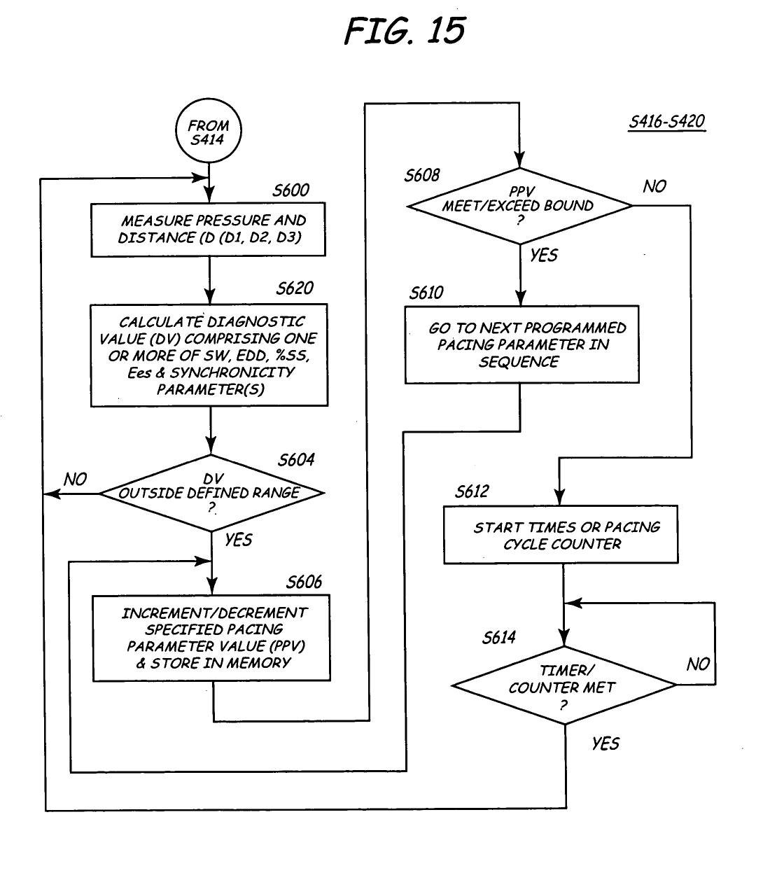

An alternative algorithm for steps S416-S420 of FIG. 7 is provided in FIG. 15. Measures of pressure P and dimension D are made periodically (even for each cardiac cycle) and are stored in device memory in step S600. The direct ventricular developed pressure P and dimensions D1, D2, and/or D3 values may be used for comparison. In addition, one or more calculated “diagnostic value” (DV) using pressure and dimension data may include, but are not limited to, stroke work (SW), end diastolic dimension (EDD), percent systolic shortening (% SS), elastance (E_{ES}) and certain “synchronicity value(s)” described further below in step S602. The -algorithm illustrated in FIG. 15 compares a current DV (which may comprise one or more of the above-listed DVs) to a defined range comprising a threshold or an upper and lower bound of the particular measured or calculated DV in step S604. The defined range threshold or boundary may be directly programmed by the physician or comprise a percentage change or other mathematical derivation (e.g. standard deviation of multiple recent measures of the test value).

The pacing parameters that are adjusted include, but are not limited to, the lower rate limit (LRL), the sensed AV delay (SAV) or paced AV delay (PAV) depending on the pacing mode, the A-A delay between delivered RA and LA pacing pulses (if operable in the system) and/or the V-V delay between delivered RV and LV pacing pulses (if operable in the system).

When any of the pacing parameter values (PPVs) are changed, a favorable therapy benefit would be expected to be provided when either or both of a change in pressure (ΔP) and a change in dimension (ΔD) exhibits an increase or no change. In regards to the other derived DVs, a favorable therapy benefit would be expected to be provided when: SW exhibits an increase or no change; EDD (two of three dimension measures) exhibits a decrease or no change; % SS (two of three dimension measures) exhibits an increase or no change; and E_{ES} exhibits an increase or no change. Thus, the threshold or range bounds for each measured DV would be programmed or set-up to fall out of these desired range. For example, SW should increase, and if SW instead falls below a threshold or lower range bound, then the PPV(s) should be adjusted to increase and bring the measured SW back into the defined range or above the threshold.

If the current observed DV(s) are found to be within the defined range in step S604, then the algorithm returns to collect another updated, current value(s) in steps S600 and S602. The current DV can be stored or used in the trend diagnostic data for later retrieval. If the current DV exceeds the threshold or lies outside of the bounds of the defined range in step S604, then the algorithm adjusts the specific PPV in step S606, and the PPV is updated and stored in memory. The PPV is checked to make sure that it is within appropriate bounds in step S608. If the PPV remains in bounds, then a programmable timer or pacing cycle counter is started in step S612. The algorithm restarts upon time-out of the programmed delay or achievement of the accumulated count of the programmed number of pacing cycles.

But, if the PPV is found in step S608 to meet or exceed the defined bound or -threshold for that pacing parameter, then the next pacing parameter in the defined or programmed sequence of pacing parameters is selected for adjustment in step S610, and it's PPV is then adjusted used in steps S606-S614. The algorithm of FIG. 15 thus adjust the defined PPVs individually or collectively in some combination, perhaps pre-specified by a programmed regimen, or in some fixed order. If the new DV(s) that are derived while pacing at the new PPVs satisfy step S604, then the IMD IPG would retain the new PPVs derived in step S606.

The time delay between a measured pressure P signal or EGM signal, e.g., a P-wave or an R-wave, or a delivered pacing pulse (Vp) and a subsequent dimension signal D during the same cardiac cycle can also provide diagnostic data that may be used to determine status and synchronicity of the ventricles of the patient as well as assist in adjustment of the pacing parameters, including the delivery of PESP stimulation. For example, the timing of the ventricular pacing Vp spike to the beginning of the movement of the individual sonomicrometer crystals (to indicate mechanical movement of the ventricle) may be measured (e.g. Vp to D1 initial movement, Vp to D2 initial movement and Vp to D3 initial movement; referenced to FIG. 1). If these time values are nearly simultaneous, then the synchronicity of the ventricle is improved (or more normal). This parameter can be measured beat-to-beat or over some time period and used as a clinical diagnostic in regards to the status of the heart failure of the patient. An increase in the standard deviation of these times or a greater difference in these times indicates a poorer synchronicity of ventricular contraction and a poorer status of the patient.

For the adjustment of pacing parameters, the timing can be measured with respect to the ventricular pacing pulse Vp to the detected movement at the different crystals. For example, in biventricular pacing with the RV pacing delivered first (adjustable AV and V-V delays), then using the D2 and D3 measures in regards to the RV pace delivery time provides time periods T2 (Vp to D2 movement, RV wall movement) and T3 (Vp to D3 movement, LV wall movement). If the difference of T2 and T3 is greater than some threshold or limit (T3−T2>threshold), then the V-V delay could be adjusted such that the site with the greater time (e.g. T3>T2) is pre-excited earlier in relation to the other site. For example, if T3=60 ms and T2=10 ms and the threshold is 20 ms, then T3−T2=50 ms and T3>T2. Thus pre-excitation of the LV site would decrease the difference. Thus, the V-V delay timing would need to be adjusted to pre-excite the LV site in relation to the RV site; e.g. if the original V-V delay timing was simultaneous (V-V delay=0 ms), then the new setting could be LV pace followed by RV pace 50 ms later. The result would be a more simultaneous contraction of the ventricles and the timing values would meet the threshold criteria. If the criteria are met, then the new value would be stored, and the algorithm reset to continue to monitor the time periods. As long as the threshold is met, then the current parameters would be maintained. If the time periods again exceeded the threshold or limit, then the interval/parameters would be adjusted again.

This adjustment would also be performed within the limits and bounds of a desired window of pressures measured using the pressure information simultaneously with the wall movement information for the adjustment of the pacing parameters. Thus, the parameter of synchronicity would operate in a similar algorithm to that depicted in FIG. 15 and described above.

Conclusion:

All patents and publications referenced herein are hereby incorporated by reference in there entireties.

It will be understood that certain of the above-described structures, functions and operations of the pacing systems of the preferred embodiments are not necessary to practice the present invention and are included in the description simply for completeness of an exemplary embodiment or embodiments. It will also be understood that there may be other structures, functions and operations ancillary to the typical operation of an AV synchronous, three or four chamber pacemaker that are not disclosed and are not necessary to the practice of the present invention. In addition, it will be understood that specifically described structures, functions and operations set forth in the above-incorporated patents and publications can be practiced in conjunction with the present invention, but they are not essential to its practice. It is therefore to be understood, that within the scope of the appended claims, the invention may be practiced otherwise than as specifically described without actually departing from the spirit and scope of the present invention.

1. In an implantable medical device, a system for monitoring the state of heart failure of the heart of a heart failure patient comprising:
pulse generating means for selectively generating and applying a pacing pulse to at least one heart chamber to effect a contraction of the heart chamber commencing a heart cycle and for selectively generating and applying an extrasystolic electrical stimulus to the at least one heart chamber at the time out of an extrasystolic escape interval to induce post-extrasystolic potentiation increasing the strength of contraction of the at least one heart chamber;
electrical signal sense means for sensing the electrical signals of the heart in X X said at least one heart chamber and providing a sense event signal signifying the contraction of the heart commencing a heart cycle;
heart chamber dimension measuring means for measuring a dimension of a heart chamber over at least a portion of a heart cycle and providing a chamber dimension value;
blood pressure measuring means for measuring blood pressure within a heart chamber over at least a portion of a heart cycle and providing a blood pressure value;
parameter deriving means for selectively enabling operation of said pulse generating means, said electrical signal sense means, said heart chamber dimension measuring means, and said blood pressure measuring means, and for periodically deriving an elastance parameter representing the slope of plotted sets of end systolic blood pressure versus end systolic chamber dimension over a plurality of heart cycles signifying the state of heart failure from selected measured values of chamber dimension and blood pressure;
means for storing the derived heart failure parameters; and
means for retrieving the stored heart failure parameters to enable a determination of the state of heart failure of the patient's heart.

2. The implantable medical device of claim 1, wherein the end systolic elastance parameter deriving means for deriving the slope of plotted sets of end systolic blood pressure versus end systolic chamber dimension over a plurality of heart cycles further comprises:
(a) means for operating said blood pressure measuring means and said heart chamber dimension measuring means to make N blood pressure (P) measurements and N dimension (D) measurements of the heart chamber at a predetermined sample rate over a series of heart cycles following a natural, intrinsic, or paced depolarization of the heart chamber;
(b) means for selecting the end systolic blood pressure (P_{ES}) measurements and end systolic distance (D_{ES}) measurements at the end systolic point in each heart cycle;
(c) means for establishing a threshold correlation coefficient R²;
(d) means for accumulating n sets of end systolic [P_{ES}, D_{ES}] data points;
(e) means for performing a linear regression of the “n” sets of [P_{ES}, D_{ES}] data points to derive the slope of the sampled data set, a sample correlation coefficient R and a sample squared correlation coefficient R²;
(f) means for comparing the sample squared correlation coefficient R² to the threshold squared correlation coefficient R²; and
(g) means for storing the derived slope as the end systolic elastance if the sample squared correlation coefficient R² exceeds the threshold squared correlation coefficient R².

3. The implantable medical device of claim 2, wherein the end systolic elastance parameter deriving means further comprises:
means operable if the sample squared correlation coefficient R² does not exceed the threshold squared correlation coefficient R² for continuously operating means (a)-(f) to develop the “n” sets of [P_{ES}, D_{ES}] data points where the oldest set of [P_{ES}, D_{ES}] data points is replaced by the newest set of [P_{ES}, D_{ES}] data points on a FIFO basis until the sample squared correlation coefficient R² exceeds the threshold squared correlation coefficient R² and for then operating means (g) for storing the derived slope as the end systolic elastance when the sample squared correlation coefficient R² exceeds the threshold squared correlation coefficient R².

4. The implantable medical device of claim 1, wherein the dimension measuring means comprises:
a first sonomicrometer piezoelectric crystal mounted to a first lead body implanted into or in relation to the first heart chamber;
a second sonomicrometer crystal mounted to a second lead body implanted into or in relation to a second heart chamber;
means for applying a drive signal and energizing one of the first and second sonomicrometer piezoelectric crystals as an ultrasound transmitter;
signal processing means coupled to the other one of the first and second sonomicrometer piezoelectric crystals operating as an ultrasound receiver that converts impinging ultrasound energy transmitted from the ultrasound transmitter through blood and heart tissue into an electrical signal;
means for measuring the time delay between the generation of the transmitted ultrasound signal and the reception of the ultrasound wave that varies as a function of distance between the ultrasound transmitter and receiver which in turn varies with contraction and relaxation of the heart chamber and providing the chamber dimension value.

5. In an implantable medical device, a system for monitoring the state of heart failure of the heart of a patient as a function of the elastance of the heart comprising:
means for defining a heart cycle;
heart chamber volume measuring means for measuring a dimension across a heart chamber over at least a portion of a heart cycle and providing a chamber dimension value;
blood pressure measuring means for measuring blood pressure within a heart chamber over at least a portion of a heart cycle and providing a blood pressure value; and
elastance parameter deriving means for deriving an elastance parameter representing the slope of plotted sets of end systolic blood pressure versus end systolic chamber volume over a plurality of heart cycles further comprising:
(a) means for operating said blood pressure measuring means and said heart chamber dimension measuring means to make N blood pressure (P) measurements and N dimension (D) measurements of the heart chamber at a predetermined sample rate over a series of heart cycles following a natural, intrinsic, or paced depolarization of the heart chamber;
(b) means for selecting the end systolic blood pressure (P_{ES}) measurements and end systolic volume (D_{ES}) measurements at the end systolic point in each heart cycle;
(c) means for establishing a threshold correlation coefficient R²;
(d) means for accumulating n sets of end systolic [P_{ES} , D_{ES}] data points;
(e) means for performing a linear regression of the “n” sets of [P_{ES}, D_{ES}] data points to derive the slope of the sampled data set, a sample correlation coefficient R and a sample squared correlation coefficient R²;
(f) means for comparing the sample squared correlation coefficient R² to the threshold squared correlation coefficient R²; and
(g) means for storing the derived slope as the end systolic elastance if the sample squared correlation coefficient R² exceeds the threshold squared correlation coefficient R².

6. The implantable medical device of claim 5, further comprising means for retrieving the stored elastance parameter to enable a determination of the state of heart failure of the patient's heart.

7. The implantable medical device of claim 5, wherein the dimension measuring means comprises:
a first sonomicrometer piezoelectric crystal mounted to a first lead body implanted into or in relation to the first heart chamber;
a second sonomicrometer crystal mounted to a second lead body implanted into or in relation to a second heart chamber;
means for applying a drive signal and energizing one of the first and second sonomicrometer piezoelectric crystals as an ultrasound transmitter;
signal processing means coupled to the other one of the first and second sonomicrometer piezoelectric crystals operating as an ultrasound receiver that converts impinging ultrasound energy transmitted from the ultrasound transmitter through blood and heart tissue into an electrical signal; and
means for measuring the time delay between the generation of the transmitted ultrasound signal and the reception of the ultrasound wave that varies as a function of distance between the ultrasound transmitter and receiver which in turn varies with contraction and relaxation of the heart chamber and providing the chamber dimension value.

8. The implantable medical device of claim 5, wherein the means for defining a heart cycle further comprises pulse generating means for selectively generating and applying a pacing pulse to at least one heart chamber to effect a contraction of the heart chamber commencing a heart cycle.

9. The implantable medical device of claim 5, wherein the means for defining a heart cycle further comprises electrical signal sense means for sensing the electrical signals of the heart in said at least one heart chamber and providing a sense event signal signifying the contraction of the heart commencing a heart cycle.

10. The implantable medical device of claim 5, wherein the end systolic elastance parameter deriving means further comprises:
means operable if the sample squared correlation coefficient R² does not exceed the threshold squared correlation coefficient R² for continuously operating means (a)-(f) to develop the “n” sets of [P_{ES}, D_{ES}] data points where the oldest set of [P_{ES}, D_{ES}] data points is replaced by the newest set of [P_{ES}, D_{ES}] data points on a FIFO basis until the sample squared correlation coefficient R² exceeds the threshold squared correlation coefficient R² and for then operating means (g) for storing the derived slope as the end systolic elastance when the sample squared correlation coefficient R² exceeds the threshold squared correlation coefficient R².

11. In an implantable medical device, a method of monitoring the state of heart failure of the heart of a patient as a function of the elastance of the heart comprising the steps of:
defining a heart cycle;
measuring a dimension of a heart chamber over at least a portion of a heart cycle and providing a chamber dimension value;
measuring blood pressure within a heart chamber over at least a portion of a heart cycle and providing a blood pressure value; and
deriving an elastance parameter representing the slope of plotted sets of end systolic blood pressure versus end systolic chamber dimension over a plurality of heart cycles further comprising the steps of:
(a) operating said blood pressure measuring means and said heart chamber volume measuring means to make N blood pressure (P) measurements and N dimension (D) measurements of the heart chamber at a predetermined sample rate over a series of heart cycles following a natural, intrinsic, or paced depolarization of the heart chamber;
(b) selecting the end systolic blood pressure (P_{ES}) measurements and end systolic dimension (D_{ES} ) measurements at the end systolic point in each heart cycle;
(c) establishing a threshold correlation coefficient R²;
(d) accumulating n sets of end systolic [P_{ES}, D_{ES}] data points;
(e) performing a linear regression of the “n” sets of [P_{ES}, D_{ES}] data points to derive the slope of the sampled data set, a sample correlation coefficient R and a sample squared correlation coefficient R²;
(f) comparing the sample squared correlation coefficient R² to the threshold squared correlation coefficient R²; and
(g) storing the derived slope as the end systolic elastance if the sample squared correlation coefficient R² exceeds the threshold squared correlation coefficient R².

12. The method of claim 11, further comprising the step of retrieving the stored elastance parameter to enable a determination of the state of heart failure of the patient's heart.

13. The method of claim 11, wherein the step of defining a heart cycle further comprises the step of selectively generating and applying a pacing pulse to at least one heart chamber to effect a contraction of the heart chamber commencing a heart cycle.

14. The method of claim 11, wherein the step of defining a heart cycle further comprises the step of sensing the electrical signals of the heart in said at least one heart chamber and providing a sense event signal signifying the contraction of the heart commencing a heart cycle.

15. The method of claim 11, wherein the end systolic elastance parameter deriving step further comprises the steps of:
continuously repeating steps (a)-(f) to develop the “n” sets of [P_{ES}, D_{ES}] data points where the oldest set of [P_{ES}, D_{ES}] data points is replaced by the newest set of [P_{ES}, D_{ES}] data points on a FIFO basis until the sample squared correlation coefficient R² exceeds the threshold squared correlation coefficient R² in step (f); and
storing the derived slope in step (g) as the end systolic elastance when the sample squared correlation coefficient R² exceeds the threshold squared correlation coefficient R² in step (f).

16. The method of claim 11, wherein the dimension measuring step comprises:
implanting a first sonomicrometer piezoelectric crystal mounted to a first lead body into or in relation to the first heart chamber;
implanting a second sonomicrometer crystal mounted to a second lead body into or in relation to a second heart chamber;
applying a drive signal and energizing one of the first and second sonomicrometer piezoelectric crystals as an ultrasound transmitter transmitting an ultrasound wave through blood and heart tissue;
sensing an electrical signal from the other one of the first and second sonomicrometer piezoelectric crystals that converts impinging ultrasound energy transmitted from the ultrasound transmitter through blood and heart tissue into an electrical signal;
e measuring the time delay between the generation of the transmitted ultrasound signal and the sensed electrical signal resulting from reception of the ultrasound wave, the time delay varying as a function of distance between the ultrasound transmitter and receiver which in turn varies with contraction and relaxation of the heart chamber; and
providing the chamber dimension value from the measured time delay.

17. In an implantable medical device, a method of monitoring the state of heart failure of the heart of a patient as a function of the elastance of the heart comprising the steps of:
implanting a first sonomicrometer piezoelectric crystal mounted to a first lead body into or in relation to the first heart chamber;
implanting a second sonomicrometer crystal mounted to a second lead body into or in relation to a second heart chamber;
implanting a blood pressure sensor into or in relation to the first heart chamber;
defining a heart cycle;
during the heart cycle measuring a dimension of a heart chamber over at least a portion of a heart cycle and providing chamber dimension values by:
applying a drive signal and energizing one of the first and second sonomicrometer piezoelectric crystals as an ultrasound transmitter transmitting an ultrasound wave through blood and heart tissue;
sensing an electrical signal from the other one of the first and second sonomicrometer piezoelectric crystals that converts impinging ultrasound energy transmitted from the ultrasound transmitter through blood and heart tissue into an electrical signal;
measuring the time delay between the generation of the transmitted ultrasound signal and the sensed electrical signal resulting from reception of the ultrasound wave, the time delay varying as a function of distance between the ultrasound transmitter and receiver which in turn varies with contraction and relaxation of the heart chamber; and
providing the heart chamber dimension value from the measured time delay;
measuring blood pressure within a heart chamber over at least a portion of a heart cycle and providing blood pressure values; and
storing the derived blood pressure and dimension values.

18. An implantable medical device for monitoring the state of heart failure of the heart of a patient as a function of the elastance of the heart comprising:
a first sonomicrometer piezoelectric crystal mounted to a first lead body implanted into or in relation to a first heart chamber;
a second sonomicrometer crystal mounted to a second lead body implanted into or in relation to a second heart chamber;
means for defining a heart cycle;
means for applying a drive signal and energizing one of the first and second sonomicrometer piezoelectric crystals as an ultrasound transmitter over at least a portion of a heart cycle;
signal processing means coupled to the other one of the first and second sonomicrometer piezoelectric crystals operating as an ultrasound receiver that converts impinging ultrasound energy transmitted from the ultrasound transmitter through blood and heart tissue into an electrical signal;
means for measuring the time delay between the generation of the transmitted ultrasound signal and the reception of the ultrasound wave that varies as a function of distance between the ultrasound transmitter and receiver which in turn varies with contraction and relaxation of the heart chamber and providing the chamber dimension value;
means for providing the heart chamber dimension value from the measured time delay;
means for measuring blood pressure within a heart chamber over at least a portion of a heart cycle and providing blood pressure values; and
means for storing the derived blood pressure and dimension values.

19. In an implantable pacing system, a method of monitoring the state of heart failure of the heart of a patient as a function of the elastance of the heart over a heart cycle and delivering a therapy to the heart comprising the steps of:
(a) implanting a first sonomicrometer piezoelectric crystal mounted to a first lead body into or in relation to the first heart chamber;
(b) implanting a second sonomicrometer crystal mounted to a second lead body into or in relation to a second heart chamber;
(c) implanting a blood pressure sensor into or in relation to the first heart chamber;
(d) pacing the heart during the heart cycle in accordance with a predetermined operating mode and parameter value;
(e) during the heart cycle, measuring a dimension of a heart chamber over at least a portion of the heart cycle and providing chamber dimension values by:
applying a drive signal and energizing the first sonomicrometer piezoelectric crystal as an ultrasound transmitter transmitting an ultrasound wave through blood and heart tissue;
sensing an electrical signal from the second sonomicrometer piezoelectric crystal that converts impinging ultrasound energy transmitted from the ultrasound transmitter through blood and heart tissue into an electrical signal;
measuring the time delay between the generation of the transmitted ultrasound signal and the sensed electrical signal resulting from reception of the ultrasound wave at the second sonomicrometer piezoelectric signal, the time delay varying as a function of distance between the ultrasound transmitter and receiver which in turn varies with contraction and relaxation of the heart chamber; and
providing the heart chamber dimension value from the measured time delay;
(f) measuring blood pressure within a heart chamber over at least a portion of a heart cycle and providing blood pressure values;
(g) employing the derived blood pressure and dimension values to derive a measure of the mechanical performance of the heart;
(h) adjusting a pacing parameter value and repeating steps (d) through (g);
(i) determining if the most recent measurement of mechanical performance derived in step (g) demonstrates an improvement in mechanical performance of the heart; and
(k) setting the pacing parameter value to the most recent measurement of mechanical performance derived in step (g) if the parameter value demonstrates an improvement in mechanical performance of the heart.

20. The method of claim 19, wherein the measure of mechanical performance derived in step (g) comprises one or more of stroke work, end diastolic dimension, percent systolic shortening, elastance, and timing relation of the dimension signal with respect to the pressure signal.

21. The method of claim 19, further comprising:
implanting a third sonomicrometer crystal mounted to a third lead body into or in relation to a third heart chamber; and
the step of measuring a dimension of a heart chamber over at least a portion of the heart cycle and providing chamber dimension values further comprises:
sensing a further electrical signal from the third sonomicrometer piezoelectric crystals that converts impinging ultrasound energy transmitted from the ultrasound transmitter through blood and heart tissue into an electrical signal;
measuring a further time delay between the generation of the transmitted ultrasound signal and the sensed electrical signal resulting from reception of the ultrasound wave at the third sonomicrometer piezoelectric crystal, the time delay varying as a function of distance between the ultrasound transmitter and receiver which in turn varies with contraction and relaxation of the heart chamber; and
providing a further heart chamber dimension value from the measured time delay.

22. The method of claim 20, wherein the measure of mechanical performance derived in step (g) comprises one or more of stroke work, end diastolic dimension, percent systolic shortening, elastance, and timing relation of the dimension signals with respect to the pressure signal.

23. In an implantable pacing system, a system for monitoring the state of heart failure of the heart of a patient as a function of the elastance of the heart over a heart cycle and delivering a therapy to the heart comprising
a first sonomicrometer piezoelectric crystal mounted to a first lead body implanted into or in relation to a first heart chamber;
a second sonomicrometer crystal mounted to a second lead body implanted into or in relation to a second heart chamber;
means for defining a heart cycle;
means for applying a drive signal and energizing one of the first and second sonomicrometer piezoelectric crystals as an ultrasound transmitter over at least a portion of a heart cycle;
signal processing means coupled to the other one of the first and second sonomicrometer piezoelectric crystals operating as an ultrasound receiver that converts impinging ultrasound energy transmitted from the ultrasound transmitter through blood and heart tissue into an electrical signal;
means for measuring the time delay between the generation of the transmitted ultrasound signal and the reception of the ultrasound wave that varies as a function of distance between the ultrasound transmitter and receiver which in turn varies with contraction and relaxation of the heart chamber and providing the chamber dimension value;
means for providing the heart chamber dimension value from the measured time delay;
means for measuring blood pressure within a heart chamber over at least a portion of a heart cycle and providing blood pressure values;
means for storing the derived blood pressure and dimension values;
means for employing the derived blood pressure and dimension values to derive a measure of the mechanical performance of the heart;
means for adjusting a pacing parameter value;
means for determining if the most recent measurement of mechanical performance demonstrates an improvement in mechanical performance of the heart; and
means for setting the pacing parameter value to the most recent measurement of mechanical performance if the parameter value demonstrates an improvement in mechanical performance of the heart.

24. The system of claim 23, wherein the measure of mechanical performance comprises one or more of stroke work, end diastolic dimension, percent systolic shortening, elastance, and timing relation of the dimension signal with respect to the pressure signal.

25. The system of claim 23, further comprising:
a third sonomicrometer crystal mounted to a third lead body into or in relation to a third heart chamber; and
the means for measuring a dimension of a heart chamber over at least a portion of the heart cycle and providing chamber dimension values further comprises:
means for sensing a further electrical signal from the third sonomicrometer piezoelectric crystals that converts impinging ultrasound energy transmitted from the ultrasound transmitter through blood and heart tissue into an electrical signal;
means for measuring a further time delay between the generation of the transmitted ultrasound signal and the sensed electrical signal resulting from reception of the ultrasound wave at the third sonomicrometer piezoelectric crystal, the time delay varying as a function of distance between the ultrasound transmitter and receiver which in turn varies with contraction and relaxation of the heart chamber; and
means for providing a further heart chamber dimension value from the measured time delay.

26. The system of claim 25, wherein the measure of mechanical performance comprises one or more of stroke work, end diastolic dimension, percent systolic shortening, elastance, and timing relation of the dimension signals with respect to the pressure signal.

27. An implantable medical device (IMD), comprising:
a first sensor to measure a dimension of a heart;
a second sensor to measure blood pressure within the heart; and
a control circuit coupled to the first and second sensors to derive at least one parameter indicative of heart failure from the dimension and the blood pressure.

28. The IMD of claim 27, and further comprising:
a delivery system coupled to the control circuit to deliver electrical stimulation to the heart; and
wherein the control circuit controls the delivery of the electrical stimulation based on the at least one parameter.

29. The IMD of claim 28, wherein the delivery system includes a circuit to deliver pacing pulses to the heart.

30. The IMD of claim 29, wherein the delivery system includes a circuit capable of delivering pacing pulses to two ventricular chambers of the heart.

31. The IMD of claim 30, wherein the first sensor comprises:
a first sonomicrometer piezoelectric crystal having a predetermined spatial relationship to a first heart chamber;
a second sonomicrometer piezoelectric crystal having a predetermined spatial relationship to a second heart chamber; and
a circuit to measure a delay between an ultrasound signal transmitted between the first and second sonomicrometer piezoelectric crystals.

32. The IMD of claim 27, wherein the control circuit includes means for deriving at least one parameter that is an elastance parameter representing the slope of plotted sets of end systolic blood pressure versus end systolic chamber dimension over a plurality of heart cycles.

33. The IMD of claim 32, wherein the means for deriving the elastance parameter comprises:
(a) means for obtaining, at a predetermined time during each of a number of cardiac cycles, a dimension measurement D from the first sensor and pressure measurement P from the second sensor; and
(b) means for deriving a slope of data points (D, P).

34. The IMD of claim 33, wherein the dimension measurement D and the pressure measurement P are both obtained at an end systolic point in each of the number of cardiac cycles.

35. The IMD of claim 30, wherein the delivery system includes a circuit capable of applying extrasystolic electrical stimulus to a chamber of the heart to induce post-extrasystolic potentiation and to thereby increase the strength of contraction of the heart chamber.

36. A method of monitoring a heart, comprising:
(a) providing a first sensor to measure a dimension of a heart;
(b) providing a second sensor to measure blood pressure within the heart; and
(c) deriving at least one parameter indicative of heart failure from the dimension and the blood pressure.

37. The method of claim 36, and further comprising delivering electrical stimulation to the heart based on the at least one parameter.

38. The method of claim 37, wherein delivering electrical stimulation comprises delivering pacing pulses to the heart.

39. The method of claim 38, wherein delivering electrical stimulation comprises delivering pacing pulses to two ventricular chambers of the heart.

40. The method of claim 36, wherein step (a) comprises:
locating a first sonomicrometer piezoelectric crystal in a predetermined position relative to a first heart chamber;
locating a second sonomicrometer piezoelectric crystal in a predetermined position relative to a second heart chamber; and
measuring a delay between an ultrasound signal transmitted between the first and second sonomicrometer piezoelectric crystals.

41. The method of claim 36, wherein step (c) includes deriving at least one parameter that is an elastance parameter representing the slope of plotted sets of end systolic blood pressure versus end systolic chamber dimension over a plurality of heart cycles.

42. The method of claim 41, and further comprising:
obtaining, at a predetermined time during each of a number of cardiac cycles, a dimension measurement D from the first sensor and pressure measurement P from the second sensor; and
deriving a slope of a line approximating interconnection of data points (D, P).
